(19) United States
(12) Patent Application Publication
(10) Pub. No.: US 2014/0155800 A1
(43) Pub. Date: Jun. 5, 2014

(54) EYE COVERINGS FOR CORNEAL HEALING AND METHODS OF USE

(71) Applicant: NexisVision, Inc., Menlo Park, CA (US)

(72) Inventors: Eugene de Juan, JR., San Francisco, CA (US); Cary J. Reich, Los Gatos, CA (US); Yair Alster, Palo Alto, CA (US); Matt Clarke, Mountain View, CA (US); Kunagmon Ashley Tuan, Mountain View, CA (US); Brian Levy, New York, NY (US)

(73) Assignee: NexisVision, Inc., Menlo Park, CA (US)

(21) Appl. No.: 14/173,516

(22) Filed: Feb. 5, 2014

Related U.S. Application Data
(63) Continuation-in-part of application No. 13/555,056, filed on Jul. 20, 2012.
Application No. 13/555,056 is a continuation of application No. 13/503,842, filed on Dec. 4, 2012.
Application No. 13/503,842 is a continuation-in-part of application No. 14/061,311, filed on Oct. 23, 2013.
Application No. 14/061,311 is a continuation of application No. 13/715,917, filed on Dec. 14, 2012, now Pat. No. 8,591,025.
(60) Provisional application No. 61/761,605, filed on Feb. 6, 2013.
(60) Provisional application No. 61/322,206, filed on Apr. 8, 2010.
(60) Provisional application No. 61/279,613, filed on Oct. 23, 2009.
(60) Provisional application No. 61/699,747, filed on Sep. 11, 2012.

(51) Int. Cl. A61F 13/12

(52) U.S. Cl. CPC A61F 13/124
USPC 602/43

(57) ABSTRACT

Methods for providing faster visual and functional recovery of patients following ocular therapy such as ocular therapy involving manipulation of the ocular tissue and/or associated with a lesion external to the optical region and ocular therapy involving incising the ocular tissue and implanting a device within the optical region are disclosed. Methods of healing a trauma wound to a cornea and methods of protecting an eye of a patient from potential injury are also disclosed. The disclosed methods comprise providing a covering to the eye of a patient wherein the covering comprises an inner portion having an inner rigidity and at least one inner radius of curvature; and an outer portion having an outer rigidity and at least one outer radius of curvature; wherein the inner rigidity is greater than the outer rigidity.

This application claims the benefit under 35 U.S.C. §119(e) of U.S. Provisional Application No. 61/761,605 filed on Feb. 6, 2013; and this application is a continuation-in-part of U.S. application Ser. No. 13/555,056 filed on Jul. 20, 2012, which is a continuation of U.S. application Ser. No. 13/503,842, filed on Dec. 4, 2012, which is the National Stage of International Application No. PCT/US2010/53975, filed on Oct. 25, 2010, which claims the benefit under 35 U.S.C. §119(e) of U.S. Provisional Application No. 61/322,206 filed on Apr. 8, 2010, and to U.S. Provisional Application No. 61/279,613 filed on Oct. 23, 2009; and this application is a continuation-in-part of U.S. application Ser. No. 14/061,311 filed on Oct. 23, 2013, which is a continuation of U.S. application Ser. No. 13/715,917 filed on Dec. 14, 2012, which claims the benefit under 35 U.S.C. §119(e) of U.S. Provisional Application No. 61/699,747 filed on Sep. 11, 2012, each of which is incorporated by reference in its entirety.

### FIELD

The present disclosure is generally directed to vision and treatment of the eye to provide improved vision. The disclosed methods and devices may, for example, provide for faster visual and functional recovery of patients following ocular therapy such as ocular therapy involving manipulation of the ocular tissue and/or associated with a lesion external to the optical region and ocular therapy involving incising the ocular tissue and implanting a device within the optical region. The methods and devices are also useful for healing a trauma wound to a cornea and for protecting an eye of a patient from potential injury. In some embodiments, the methods comprise providing a covering to the eye of a patient wherein the covering comprises an inner portion having an inner rigidity and at least one inner radius of curvature and an outer portion having an outer rigidity and at least one outer radius of curvature, wherein the inner rigidity is greater than the outer rigidity.

### BACKGROUND

The eye includes several tissues that allow patients to see. The cornea of the eye is an anterior tissue of the eye that is clear in healthy eyes and refracts incident light so as to form an image on the retina. The retina is a posterior tissue of the eye that senses light from the image formed thereon and transmits signals from the image to the brain. The cornea includes an outer layer of tissue, the epithelium, which protects the underlying tissues of the cornea, such as Bowman's membrane, the stroma, and nerve fibers that extend into the stroma and Bowman's membrane. The healthy eye includes a tear film disposed over the epithelium. The tear film can smooth small irregularities of the epithelium so as to provide an optically smooth surface. The tear film is shaped substantially by the shape of the underlying epithelium, stroma, and Bowman's membrane, if present. The tear film comprises a liquid that is mostly water and includes additional components such as mucoids and lipids. The nerve fibers of the cornea provide sensation to promote blinking that can serve to cover the cornea with the tear film. The nerve fibers also sense pain so that one will avoid trauma to the cornea and also avoid direct contact of an object to the cornea so as to protect this important tissue.

It has been recognized that coverings may be useful in enhancing visual and functional recovery in other ocular therapies. Manipulation of the cornea such as in surgical procedures generally results in edema caused by excess hydration. Corneal swelling associated with edema can degrade vision due, for example, to light scattering and haze. Edema is generally caused by the compromise of the membranes of the eye responsible for maintaining deturgescence of the cornea. Visual acuity is compromised until the membranes heal and corneal deturgescence is restored. For example, in corneal inlay surgery full visual recovery is not fully restored until about two weeks following the surgery.

Other complications associated with corneal manipulation include transient surface irregularities and leakage from a surgical incision. Following corneal manipulation, the ocular membranes and/or inlays may not form smooth surfaces. The transient irregularities, which resolve over time, can cause suboptimal visual acuity during the first several hours or days following surgery. Also, incisions in the cornea may not fully seal to cause loss of visual acuity or may even necessitate follow-on treatment. For example, following cataract surgery, about 25% of patients experience leakage from the sutured incision.

Coverings and the use of coverings having an inner portion having an inner rigidity and at least one inner radius of curvature; and an outer portion having an outer rigidity and at least one outer radius of curvature; wherein the inner rigidity is greater than the outer rigidity have been shown to be useful in restoring visual acuity and increasing post-therapy comfort following ocular procedures such as photorefractive keratectomy (PRK) and laser assisted in-situ keratomileusis (LASIK). For example, U.S. application Ser. No. 13/555,056 filed on Jul. 20, 2012, describes the use of coverings for the treatment of PRK and U.S. application Ser. No. 13/715,917 filed on Dec. 14, 2012, describes the use of coverings for the treatment of LASIK. The ability of such coverings to enhance visual acuity and to reduce complications following PRK and LASIK procedures can be effective in optimizing the outcome in other ocular therapies including wound healing.

Methods and devices for decreasing the time to optimal visual outcome following ocular therapy and for minimizing complications following corneal manipulation, especially during the first several hours following ocular therapy are desired. Devices that when applied to the eye prior to a potential injury, can serve a protective function are also desired.

### SUMMARY

In a first aspect, methods of treating an eye of a patient following an ocular therapy are provided, the eye comprising ocular tissue including a cornea having an anterior surface and an optical region used for imaging with the eye, an epithelium, and a sclera, the ocular therapy involving manipulation of the ocular tissue and associated with a lesion external to the optical region, the method comprising: providing a covering comprising an inner portion characterized by an inner rigidity, and comprising an upper surface and a lower surface; and an outer portion characterized by an outer rigidity, and comprising an upper surface and a lower surface; wherein the inner rigidity is greater than the outer rigidity; following the ocular therapy, applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In a second aspect, methods of treating an eye of a patient following an ocular therapy are provided, the eye comprising ocular tissue including a cornea having an anterior surface and an optical region used for imaging with the eye, an epithelium, and a sclera, the ocular therapy involving incising the ocular tissue and implanting a device within the optical region, the method comprising: providing a covering comprising an inner portion characterized by an inner rigidity, and comprising an upper surface and a lower surface; and an outer portion characterized by an outer rigidity, and comprising an upper surface and a lower surface; wherein the inner rigidity is greater than the outer rigidity; following the ocular therapy, applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In a third aspect, methods of treating an eye of a patient following cataract surgery or corneal inlay surgery are provided, the eye comprising a cornea comprising an anterior surface, a stroma, an epithelium, and a sclera, the anterior surface of the cornea characterized by a post-surgical profile, the method comprising: providing a covering comprising an inner portion comprising a first lower surface characterized by at least one inner radius of curvature, wherein the at least one inner radius of curvature comprises a radius of curvature less than a radius of curvature of the post-surgical profile of the anterior surface of the cornea, and the inner portion is characterized by a substantially uniform thickness; and an outer portion comprising a second lower surface characterized by at least one outer radius of curvature; and applying the covering, after the cataract surgery or the corneal inlay surgery and prior to complete healing of the eye therefrom, wherein the covering is applied against the eye so that the covering flexes, with the inner portion at least partially disposed along and deforming so that an upper surface of the inner portion opposite the first lower surface of the inner portion optically conforms to the anterior surface of the cornea and at least a part of the outer portion engages the eye along the epithelium, the sclera, or a combination thereof.

In a fourth aspect, methods of treating an eye of a patient following ocular surgery involving manipulation of the cornea are provided, the method comprising applying a covering to the eye of the patient following ocular surgery, wherein following treatment with the method, at least about 50% of a population of patients see 20/20 or better within about 5 minutes following application of the covering.

In a fifth aspect, methods of healing a trauma wound to a cornea of an eye of a patient are provided, the eye comprising a cornea having an anterior surface, an epithelium, and a sclera, the method comprising: providing a covering comprising an inner portion characterized by an inner rigidity, and comprising an upper surface and a lower surface; and an outer portion characterized by an outer rigidity, and comprising an upper surface and a lower surface; wherein the inner rigidity is greater than the outer rigidity; applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In a sixth aspect, methods of protecting an eye of a patient from a potential injury are provided, the eye comprising a cornea having an anterior surface, an epithelium, and a sclera, the method comprising: providing a covering comprising an inner portion characterized by an inner rigidity, and comprising an upper surface and a lower surface; and an outer portion characterized by an outer rigidity, and comprising an upper surface and a lower surface; wherein the inner rigidity is greater than the outer rigidity; applying the covering against the eye prior to the potential injury so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

### BRIEF DESCRIPTION OF THE DRAWINGS

The drawings described herein are for illustration purposes only. The drawings are not intended to limit the scope of the present disclosure.

FIG. 1 shows a schematic cross-section of a human eye.

FIG. 2 shows a schematic cross-section of a portion of an eye prior to photorefractive surgery.

FIG. 3A shows a schematic cross-section of a portion of an eye during cataract surgery.

FIG. 3B shows a schematic cross-section of a portion of an eye following LASIK.

FIG. 4A shows a schematic cross-section of a simplified covering provided by the present disclosure.

FIG. 4B shows a schematic cross-section of a portion of the eye shown in FIG. 3B following LASIK with the covering shown in FIG. 4A applied to the anterior surface of the cornea.

FIG. 4C shows a schematic cross-section of a portion of the eye shown in FIG. 3A following cataract surgery with the covering shown in FIG. 4A applied to the anterior surface of the cornea.

FIG. 5 is a graph showing the improvement in the uncorrected visual acuity (UCVA) and the best corrected visual acuity (BCVA) with time following LASIK surgery.

FIG. 6 is a graph showing the change in the refractive vision component with time in terms of spherical equivalent (D) following LASIK surgery.

FIG. 7A shows a plot of the sagittal curvature of a cornea one hour following LASIK surgery measured using a corneal topographer.

FIG. 7B shows the elevation profile of a cornea one hour following LASIK surgery measured using a corneal topographer.

FIG. 8A shows a cross-sectional view of a cornea before LASIK surgery.

FIG. 8B shows a cross-sectional view of a cornea following LASIK surgery.

FIG. 9 shows a cross-sectional view of a covering according to certain embodiments of the present disclosure.

FIG. 10 shows a cross-sectional view of a covering according to certain embodiments of the present disclosure.

FIG. 11 is a graph of the monocular UCVA following LASIK surgery in a population of patients wearing a covering provided by the present disclosure (Durrie NXV LASIK) and in a population of patients without the treatment (Durrie LASIK).

FIGS. 12A-12F are histograms showing the percent of patients having a certain visual acuity or better at various times following LASIK surgery. The visual acuity of a population of patients wearing a covering provided by the present disclosure (NXV) is compared to that of a population of patients not wearing a covering (not treated). The histograms represent measurements made immediately following surgery (FIG. 12A), 30 minutes following surgery (FIG. 12B), 1 hour following surgery (FIG. 12C), 2 hours following surgery (FIG. 12D), 4 hours following surgery (FIG. 12E), and 1 day following surgery (FIG. 12F).

FIG. 13 is a graph of the frequency-dependent monocular mesopic contrast sensitivity (log CS) for a population of patients prior to LASIK surgery (pre-op), following LASIK surgery with various surgical platforms (Alcon, AMO, and Ziemer) and without post-surgical treatment, and following LASIK using the Alcon platform and with post-LASIK patients wearing a covering provided by the present disclosure following surgery (NXV).

FIG. 14 shows the relationship between contrast sensitivity and visual acuity and also the typical contrast sensitivity for normal vision (contrast sensitivity curve).

FIG. 15 is a graph showing the decrease in stromal edema in eyes for a population of patients following LASIK surgery without wearing a covering (LASIK benchmark) and for a population of patients wearing a covering provided by the present disclosure (NXV LASIK).

FIG. 16A shows (a) a histogram reflecting the percent of patients (n=10) experiencing various levels of overall discomfort with time following LASIK surgery; and (b) a table of the mean overall discomfort score based on the histogram, in a population of LASIK patients without treatment.

FIG. 16B shows (a) a histogram reflecting the percent of patients (n=8) experiencing various levels of overall discomfort with time following LASIK surgery; and (b) a table of the mean overall discomfort score based on the histogram, in a population of LASIK patients wearing a covering provided by the present disclosure.

FIG. 17A shows (a) a histogram reflecting the percent of patients (n=10) experiencing various levels of outdoor photophobia with time following LASIK surgery; and (b) a table of the mean outdoor photophobia score based on the histogram, in a population of untreated LASIK patients.

FIG. 17B shows (a) a histogram reflecting the percent of patients (n=8) experiencing various levels of outdoor photophobia with time following LASIK surgery; and (b) a table of the mean outdoor photophobia score based on the histogram, in a population of patients wearing a covering provided by the present disclosure.

FIG. 18A shows (a) a histogram reflecting the percent of patients (n=10) experiencing various levels of burning sensation with time following LASIK surgery; and (b) a table of the mean burning sensation score based on the histogram, in a population of untreated LASIK patients.

FIG. 18B shows (a) a histogram reflecting the percent of patients (n=8) experiencing various levels of burning sensation with time following LASIK surgery; and (b) a table of the mean burning sensation score based on the histogram, in a population of LASIK patients wearing a covering provided by the present disclosure.

FIG. 19 shows a histogram of the percent of patients able to text at various times following LASIK surgery without treatment (LASIK) and with treatment (NXV LASIK) according to methods provided by the present disclosure.

FIG. 20 shows a histogram of the percent of patients who feel comfortable driving at various times following LASIK surgery without treatment (LASIK) and with treatment (NXV LASIK) according to methods provided by the present disclosure.

FIG. 21 is a scatter chart showing the monocular uncorrected visual acuity (UCVA) at various times following LASIK surgery for patients wearing coverings having central (3 mm) curvatures between about −1.0D and −3.0D relative to the central curvature of the cornea.

FIG. 22 is a scatter chart showing the decrease in uncorrected visual acuity (UCVA) after coverings having different central (3 mm) curvatures are removed from a patient's eye.

FIG. 23 is a graph showing the UCVA following photorefractive keratectomy (PRK) surgery in a population of patients (n=6) wearing a covering having a central curvature about the same as the central curvature of the cornea (on-K), and for a population of patients wearing a covering having a central curvature from about 1.0D to about 2.5D flatter than the curvature of the cornea (flat-K), e.g., having a curvature −1.0D to about −2.5D that of the cornea.

FIG. 24 is a scatter chart showing the UCVA at various times following LASIK surgery for a population of patients (Durrie n=89) wearing coverings having a curvature about 1.0D to about 3.0D flatter (−1.0D to −3.0D) than the curvature of the post-PRK cornea.

FIG. 25 is a scatter chart showing the discomfort two days (48 hours) following PRK surgery in a population of patients (Durrie n=89) wearing coverings having different mid-periphery (5 mm) curvatures.

FIG. 26 is a scatter chart showing the area of epithelial defects two days (48 hours) following PRK surgery in patients (Durrie n=89) wearing coverings having different mid-periphery (5 mm) curvatures from about 0D to about −6D.

FIG. 27 is a scatter chart showing the area of epithelial defects two days (48 hours) following PRK surgery in patients (Durrie n=89) wearing coverings having different mid-periphery (5 mm) curvatures from about 0D to about −3D.

FIG. 28A shows a schematic cross-section of a cornea following LASIK surgery with an edematous flap having topographical irregularities.

FIG. 28B shows a schematic cross-section of a cornea following LASIK surgery with a covering provided by the present disclosure applied over the anterior surface of the cornea and smoothing the edematous flap.

FIG. 29 shows a photograph of a corneal cross-section following LASIK surgery with a covering applied to the anterior surface of the cornea.

FIG. 30 shows a photograph of a cross-section of a cornea following PRK in which a bandage contact lens has been applied to the anterior surface of the cornea. The inset shows a magnified image of the interface between healthy epithelium and the ablated region of the cornea.

FIG. 31 shows a photograph of a cross-section of a cornea having an ablated stroma two days following PRK surgery. The covering applied to the anterior surface of the cornea is shown to form a lenticular volume between the inner surface of the covering and the ablated stroma and conforming to the healthy, non-ablated epithelium.

FIG. 32 is a graph showing improvement in monocular UCVA (log MAR) during the first four hours following LASIK without treatment (Durrie n=104) and with a covering applied to the cornea (NXV n=60). The differences immediately following surgery, and at 30 minutes, 1 hour, and 2 hours are statistically significant (p<0.0001).

FIG. 33 shows histograms comparing the percent of patients having a UCVA of 20/20 or better during the first few hours following LASIK without treatment (LASIK n=52) and wearing a covering provided by the present disclosure (NXV n=30).

FIG. 34 shows histograms comparing the percent of patients having a UCVA of 20/25 or better during the first few hours following LASIK without treatment (LASIK n=52) and wearing a covering provided by the present disclosure (NXV n=30).

FIG. 35 shows histograms comparing the percent of patients having a particular binocular visual acuity (high contrast functional visual acuity) during immediately following LASIK without treatment (LASIK n=52) and wearing a covering provided by the present disclosure (NXV n=30).

FIG. 36 shows histograms comparing the percent of patients able to text at a different times following LASIK without treatment (LASIK n=56) and wearing a covering provided by the present disclosure (NXV n=27).

FIG. 37 shows histograms comparing the percent of patients able to drive at a different times following LASIK without treatment (LASIK n=57) and wearing a covering provided by the present disclosure (NXV n=43).

FIG. 38 is a graph comparing the contralateral UCVA (log MAR) during the first 24 hours following LASIK in patients (n=5) wearing a bandage contact lens in one eye (control) and wearing a covering provided by the present disclosure in the other eye (NXV).

FIG. 39 shows a histogram of the contralateral overall level of discomfort associated with the eye wearing a covering provided by the present disclosure for a group of patients (n−10) following LASIK.

FIG. 40 shows a histogram of the contralateral overall level of discomfort associated with the eye wearing a bandage contact lens for the group of patients of FIG. 39 (n−10) following LASIK.

FIG. 41 is a graph comparing the contralateral discomfort scores based on a weighted analysis of discomfort criteria in a group of patients wearing a bandage contact lens (n=10) in one eye and wearing a covering provided by the present disclosure (n=10) in the other eye. The discomfort scores were derived from the results presented in FIG. 39 and in FIG. 40.

FIG. 42 is a histogram showing patient (n=10) preference for either a bandage lens or a covering based on a contra-lateral comparison during the first 24 hours following LASIK.

FIG. 43 is a graph comparing the best corrected visual acuity (BCVA) (log MAR) during the first 3 days following LASIK in a group of patients (n=25) wearing a bandage contact lens in one eye and a covering in the other eye. The results are statistically significant (p<0.0001) at all times post-PRK.

FIG. 44 is a graph comparing edema during the first 72 hours following PRK in a group of patients (n=17) wearing a bandage contact lens and a group of patients (n=10) wearing a covering.

Reference is now made in detail to embodiments of the present disclosure. While certain embodiments of the present disclosure are described, it will be understood that it is not intended to limit the embodiments of the present disclosure to the disclosed embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

“Engaging” and “engagement” refers to the ability of a covering to attach to ocular tissue in a matter that resists movement of the covering and seals the cornea.

“Optical region” refers to the region of the eye or a covering that is used for vision. In general, the optical region encompasses the central 3 mm portion of the eye or covering.

“Optically conforms” refers to the capability of the inner surface of a covering provided by the present disclosure to provide a smooth optical surface that substantially matches the overall shape of the cornea. For example, a covering or a portion of a covering that optically conforms to the anterior surface of the cornea may or may not directly contact corneal tissue such as the stroma and/or the epithelium. In certain embodiments, a covering that optically conforms to the anterior surface of the cornea bridges epithelial defects such that in the region of the defects, the inner surface of the covering does not physically contact the cornea. In certain embodiments, the inner portion of a covering is configured to optically conform to the optical region of the anterior surface of the cornea, and the outer portion of a covering is configured to physically conform to the non-optical region of the cornea.

“Physically conforms to” refers to the capability of the inner surface of the covering or at least portions of the inner surface of a covering to contact adjoining ocular tissue.

“Resisting movement” refers to the capability of a covering to maintain its position on the cornea during blinking and conditions of normal use.

“Sealing” refers to the ability of a covering provided by the present disclosure to substantially prevent egress and ingress, i.e., transport, of fluids including water, ocular fluid, and tear fluid from and to the cornea. A covering is formed using a material such as a silicone polymer or a silicone hydrogel that has a low water content and is characterized by a low water or ion permeability.

“Substantially conforms to,” “conforms to” and “physically conforms to” refer to the capacity of a device to physically contact the anterior surface of the cornea. Coverings provided by the present disclosure are configured such that when applied to the anterior surface of the cornea, the anterior surface of the cornea substantially conforms to the inner surface of the covering.

As used herein, a covering refers to an ophthalmic device that covers an eye of a patient and that may or may not provide refractive vision correction.

As used herein, an on-K fit of a covering encompasses fitting a covering to the flattest meridian of the cornea and the on-K fit can be flatter than the flattest meridian within about 1.5D. For example, for a cornea having keratometer values (K-value) of about 44D axis 90 and 43D axis 180, the on-K fit provides a covering having a curvature corresponding to an optical power within a range from about 43D to about 41.5D for the region of the eye measured. The on-K fit as described herein can allow for faster visual recovery and for minimization of discomfort following ocular therapy.

The optical power of the cornea in Diopters (D) can be related to the radius of curvature R by the formula D=(1.3375−1)/R, where 1.3375 corresponds to the index of refraction of the aqueous humor and R corresponds to the radius of curvature of the anterior surface of the cornea. The curvature of the cornea is inversely related to the radius of curvature R such that as the radius of curvature increases the curvature of the cornea decreases and such that as the radius of curvature decreases the curvature of the cornea increases.

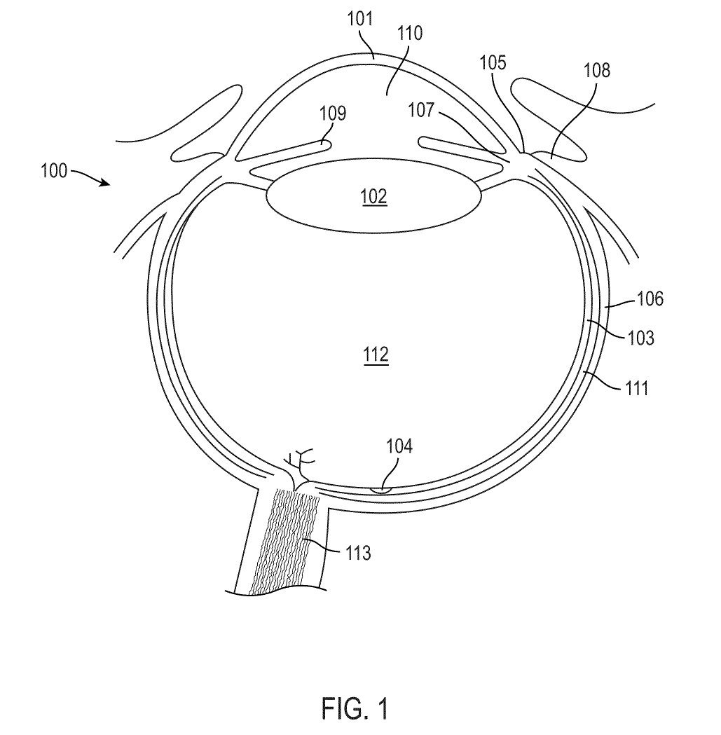

FIG. 1 shows a cross-section of a human eye 100. The eye has a cornea 101 and a lens 102 configured to form an image on the retina 103, where the image can form on a fovea 104 corresponding to high visual acuity. The cornea 101 can extend to a limbus 105 of the eye, and the limbus 105 can connect to a sclera 106 of the eye. The eye 100 has a pars plana 107 located near limbus 105. A conjunctiva 108 of the eye can be disposed over the sclera 106. The lens 102 can accommodate to focus on an object seen by a patient. The eye 100 has an iris 109 that defines a pupil 110 that may expand and contract in response to light. The eye also comprises a choroid 111 disposed between the sclera 106 and the retina 103. The eye has a vitreous humor 112 extending between the lens 102 and the retina 103. The retina 103 senses light of an image and converts the light image to neural signals that are processed and transmitted along an optic nerve 113 onto the brain of the patient.

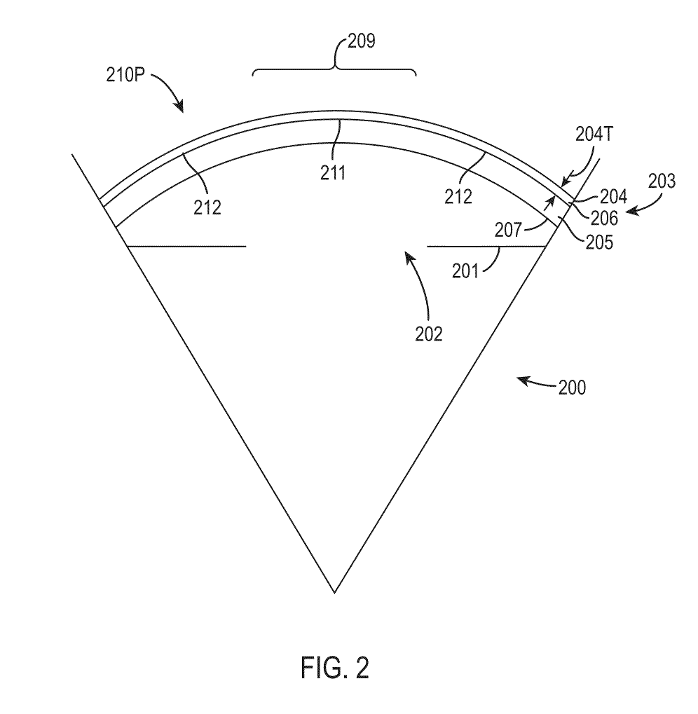

FIG. 2 shows a cross-section of an eye before ocular therapy. The eye 200 comprises an iris 201 that defines a pupil 202, through which light passes such that the patient can see. Cornea 203 includes an epithelium 204 disposed over a stroma 205. The epithelium 204 comprises a thickness 204T that can be about 50 μm. A tear liquid (not shown) covers the anterior surface of epithelium 204. In at least humans, primates, and some birds, a Bowman's membrane 206 is disposed between epithelium 204 and stroma 205. Bowman's membrane 206 comprises an acellular substantially collagenous tissue with a thickness of about 5 μm to 10 μm. Stroma 205 comprises a substantially collagenous tissue with keratocytes disposed therein. In some animals, Bowman's membrane may be absent and the epithelium may be disposed adjacent to the stromal layer. An endothelium 207 is disposed beneath stroma 205. Endothelium 207 comprises a layer of cells that pump water from cornea 203 toward iris 201. An optical region 209 of the epithelium 204 and stroma 205 is aligned with pupil 202 so as to be used by the eye when optically imaging. At least some portion of the epithelium and the stroma, as well as the surrounding sclera is outside the region used for optical imaging. The anterior surface of the cornea 203 including stroma 205 and epithelium 204 defines a pre-therapy ocular profile 210P.

In a normal healthy eye, epithelium 204 is disposed across cornea 203 and functions as a protective layer. Epithelium 204 covers nerves of the cornea and minimizes the flow of water from the tear film of the eye to into the stroma 205. Epithelium 204 in most human patients can be about 40 microns to 60 microns thick, for example about 50 microns. When epithelium 204 is intact, endothelium 207 can pump water from stroma 205 and maintain hydration in the cornea at a proper level. The mechanism by which the stroma of the cornea remains properly hydrated can be referred to as deturgescence. Deturgescence of the cornea can be important because excess hydration of the cornea can result in swelling of the cornea and light scattering, or haze, that can degrade vision. The total thickness of normal cornea 203 from endothelium 207 to the tear liquid overlying epithelium 204 in most human patients can be from about 400 microns to 600 microns. A healthy cornea with normal hydration comprises about 80% to 85% water. Edema of the cornea due to swelling of the cornea can increase, the thickness of the cornea.

A number of ocular therapies can be undertaken to enhance or restore visual acuity. Ocular therapies may involve manipulation of the ocular tissue and can be associated with a lesion external to the optical region. In certain embodiments, ocular therapies involve incising an ocular tissue and implanting a device within the optical region. In certain embodiments, ocular therapies involve ablating at least a portion of the stroma and/or epithelium. Ocular therapies include, for example, cataract surgery including phacoemulsification, conventional extracapsular cataract extraction, and intracapsular cataract extraction; glaucoma surgery including laser trabeculoplasty, irdotomy, irdectomy, sclerotomy, goniotomy, drainage implant surgery, and canaloplasty; corneal surgery including corneal transplant surgery, penetrating keratopalsty, keratoprosthesis, pterygium excision, corneal tattooing, and osteo-ondonto-keratoprosthesis; and photorefractive therapy including photorefractive keratectomy (PRK) and laser-assisted in-situ keratomileusis (LASIK). Ocular therapy may also involve treating a wound to the eye, wherein the treatment may or may not involve ocular surgery. In certain embodiments, ocular therapy is selected from cataract surgery, corneal inlay surgery, corneal transplant surgery, and treatment of an ocular trauma wound. In certain embodiments, ocular therapy comprises incising the cornea and/or perforating the cornea at a site external to the optical region.

In certain embodiments, coverings provided by the present disclosure may be used to treat corneal inlay surgery or corneal onlay surgery. Corneal inlays and onlays are tiny lenses or other optical devices inserted into the cornea to reshape the front surface of the eye, i.e., the anterior surface of the cornea, to improve vision and in some cases can resemble small contact lenses. The primary use of current corneal inlays is to improve near vision and to address presbyopia. In some cases, corneal inlay surgery can be combined with photorefractive surgery such as LASIK to correct both presbyopia and common refractive errors such as nearsightedness, farsightedness, and/or astigmatism. Because corneal inlays are implanted within the tissue of the cornea, this type of refractive surgery is less invasive than phakic intraocular lens procedures that involve larger implantable lenses that are placed deeper in the eye such as in front of or behind the pupil. Corneal inlay surgery involves providing a stromal flap and inserting a corneal inlay within the flap. In contrast, corneal onlays do not require a thin flap, as occurs with inlays, but instead are placed in an artificially created pocket under the epithelium. This pocket then holds the corneal onlay in place until the disturbed epithelial cells grow back to cover the device.

In certain embodiments, coverings provided by the present disclosure may be used to treat cataract surgery In certain embodiments, ocular therapy comprises cataract surgery. Cataract surgery involves the removal and replacement of the natural lens of the eye that has developed opacificaiton, which is referred to as a cataract. Following surgical removal of the natural lens, an artificial intraocular lens implant is inserted or implanted. The most common types of cataract surgery extraction are phacoemulsification and conventional extracapsular cataract extraction. In phacoemulsification, an ultrasonic device is used to emulsify the natural lens material. Following emulsification, a small (3-5 mm) incision is made in the cornea and/or sclera outside the optical region and the emulsified lens and cortical material surrounding the lens is aspirated. Alternatively, in conventional extracapsular cataract extraction the entire natural lens is removed while leaving the elastic lens capsule intact to allow implantation of an intraocular lens. Removing the natural lens involves manual expression of the lens through a large (10 mm to 12 mm) incision made in the cornea or sclera, After the natural lens is removed, an intraocular lens is implanted into the eye, either through a small (e.g., 1.8 mm to 2.8 mm) incision using a foldable intraocular lens, or through an enlarged incision. The small size of the wound in phacoemulsification usually allows closure without the use of stitches, whereas extracapsular cataract extraction usually requires stitching.

In certain embodiments, coverings provided by the present disclosure may be used to treat corneal transplantation surgery Corneal transplantation therapies include, for example, penetrating keratoplasty, lamellar keratoplasty, deep anterior lamellar keratoplasty, and endothelial keratoplasty. In these surgical procedures one or more corneal tissues or in some cases the entire cornea is removed and replaced with a corresponding tissue or cornea. Corneal transplantation is often used to treat pseudophakic bullous keratopathy, keratoconus, corneal degeneration, keratoglobus, dystrophy as well as scaring due to keratitis or trauma.

In general, ocular therapies such as cataract surgery, corneal inlay surgery, and corneal transplant surgery can be distinguished from ocular therapies involving manipulation only to the optical region of the cornea or primarily to the optical region of the cornea. In the former ocular therapies, which can be considered implantation surgeries in that a device is implanted into an ocular tissue as an adjunct or as a replacement for an ocular tissue that is removed, the procedures involve manipulation of ocular tissue external to the optical region as well as to the optical region itself. The latter therapies are exemplified by refractive surgeries in which the optical region of the cornea is sculpted to correct refractive visual error. Examples of refractive surgeries include, for example, PRK and LASIK. Ocular therapies involving manipulation of the optical region of the cornea are encompassed to the extent that the therapy also involves manipulation of ocular tissue external to the optical region. For example, LASIK involves making an incision in the stroma external to the optical region to form a flap. The flap is then lifted back to expose the stroma, which is then ablated using a laser to provide a shape for refractive correction. Furthermore, ocular manipulation involving tissue external to the optical region and photorefractive surgery involving manipulation of tissue within the optical region can be combined. For example, corneal inlay surgery and associated photorefractive surgery such as LASIK surgery can be combined.

Following ocular therapy, defects to the anterior surface of the cornea may be present. Depending on the type of ocular therapy, portions of the epithelium may be removed, exposed regions of the stroma may be present, and/or there may be incisions, holes and/or perforations through portions of the epithelium and stroma. In certain cases, the defects in the anterior surface of the cornea may be situated in non-optical portions of the cornea, located within the optical portion, or may be located in both the optical and non-optical portions of the cornea. In certain embodiments, a covering is configured to extend over the defects to the anterior surface of the cornea to provide a lenticular region that maintains a physiological environment that promotes epithelial growth. In such embodiments, the surface of the covering can smooth the anterior surface of the cornea, seal the defect, hold ocular tissue in pace, resist motion of ocular tissue, and/or promote growth and healing of ocular tissue.

In certain embodiments, coverings provided by the present disclosure when applied to an eye of a patient following ocular therapy speed healing of ocular defects. Ocular defects include incisions and perforations of the cornea and/or other ocular tissue. An ocular defect may be associated with manipulation of the cornea and/or other ocular tissue outside the optical region of the eyes. An ocular defect may be associated with manipulation of the cornea and/or other ocular tissue outside the optical region of the eye and within the optical region of the eye. For example, cataract surgery can involve manipulation of the cornea such as by an incision outside the optical region of the eye and manipulation of the lens within the optical region of the eye by extracting the old lens and replacement with a new lens.

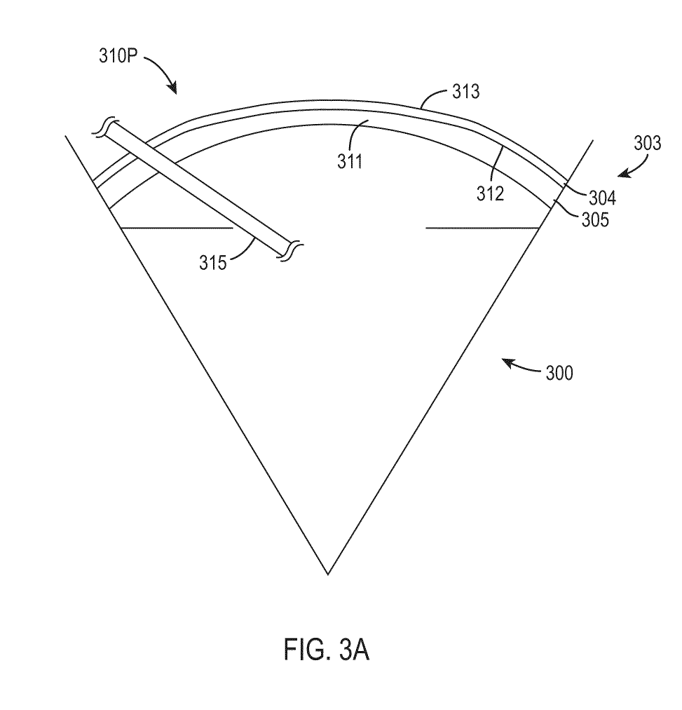

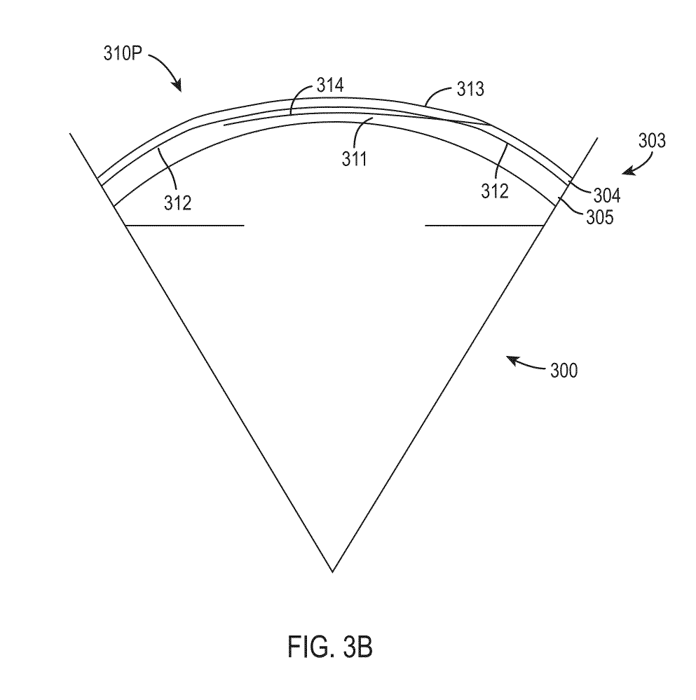

FIG. 3A shows the portion of the eye 300 illustrated in FIG. 2 during cataract surgery. During cataract surgery a cataract treatment probe 315 is inserted into the eye through an incision or wound outside the optical region. The eye shown in FIG. 3A includes center portion 311 of the stroma 305 and epithelium 305. FIG. 4C shows an eye following cataract surgery with a cover provided by the present disclosure substantially conforming to the anterior surface of the cornea. As shown, the peripheral portion of the covering extends over incision or wound 316, to seal incision or wound 316 and to facilitate healing. In certain embodiments, the incision or wound from the cataract surgery may be outside the optical region, within the optical region, or both within and without the optical region. In certain embodiments, the rigid central portion of the covering and/or the flexible peripheral portion of the covering may overly the cataract incision or wound.

In certain embodiments, coverings provided by the present disclosure may be used to treat cross-linking therapy. Corneal cross-linking is a technique that strengths the chemical bonds in the cornea and thereby resist irregular changes to the corneal shape known as ectasia. Ectatic changes are typically marked by corneal thinning and an increase in the anterior and/or posterior curvatures of the cornea and can lead to myopia and astigmatism. Ectasia is associated with keratoconus and post-refractive surgery ectasia, although other applications for corneal collagen cross-linking include pellucid marginal degeneration, iatrogenic keratectasia, prevention of keratectasia, bullous keratopathy, microbial keratitis, corneal ulceration, donor tissue modification prior to keratoplasty, and as an adjutant to orthoeratology. Corneal cross-linking may also be combined with PRK, LASIK, and other ocular therapies.

The progression of ectasia can be mitigated by strengthening corneal tissue by increasing the cross-linking density of the collagen fibers making up the cornea. This can be accomplished by applying a photosensitizer such as riboflavin to the cornea followed by irradiation with UV light. A portion of the epithelium is generally removed prior to application of the photosensitizer to the cornea.

While the foregoing has focused on ocular therapies associated with intentional manipulation of the eye, it can be appreciated that coverings and methods of using the coverings can also be useful in treating other injuries to the eyes such as, for example, the treatment of trauma wounds. Trauma to the eye can also cause edema and compromise the interfaces between the various ocular tissue. Thus, in addition to post-surgical methods, coverings provided by the present disclosure are useful in healing trauma wounds to the eye. Trauma includes, for example, physical trauma such as blunt trauma and penetrating trauma, chemical trauma, blast injury, burn, and psychological trauma. Treatment of a trauma wound may involve surgical procedures such as removing an embedded physical object or removing scar tissue. To the extent that the trauma produces edema and optical irregularities, application of a covering will lead to faster visual recovering and by stabilizing the involved ocular tissue, and accelerate healing. Trauma may also cause defects to ocular tissue including to the anterior surface of the cornea and involve the epithelium and/or stroma and may cause damage to internal ocular tissue. Wound healing thus includes healing wounds associated with physical damage to ocular tissue not necessarily caused by surgical procedures.

In certain embodiments, coverings provided by the present disclosure may be used to treat edema. Corneal edema is excess hydration or accumulation of fluid in the cornea causing the cornea to swell. Corneal edema may be caused, for example, by dehydration, viral infection, an endothelial disorder, impairment of the endothelial layer, damaged Descemet's membrane, traumatic injury, increased ocular pressure, toxins, topical or systemic medication, or ocular surgery.

In certain embodiments, coverings provided by the present disclosure may be used to treat photorefractive therapy such as, for example, PRK and LASIK. Refractive eye surgery is used to improve the refractive state of the eye and includes procedures such as, for example, automated lamellar keratoplasty (ALK), laser assisted in-situ keratomileusis (LASIK), photorefractive keratectomy (PRK), laser assisted sub-epithelium keratomeleusis (LASEK), EPI-LASIK, radial keratotomy, mini-asymmetric radial keratotomy, arcuate kertotomy, limbal relaxing incisions, thermal keratoplasty, laser thermal keratoplasty, intrastomal corneal ring segment removal, and phakic intraocular lens implantation. Following any of these procedures there is a period of time before optimal vision is restored. For example, in LASIK, optimal vision is typically achieved within about 24 hours following surgery. During this recovery period, in addition to sub-optimal visual acuity, a patient may experience discomforts such as photophobia or light sensitivity and/or a burning sensation. Methods for reducing the time to achieve optimal vision and for reducing or eliminating discomfort associated with refractive eye surgery are desired.

PRK is a surgical procedure in which typically a laser is used to shape the stroma to correct for photorefractive error. In the process, the epithelium overlying the portion of the ablated stroma is removed to form an epithelial defect.

LASIK is a surgical procedure used to correct refractive vision errors such a myopia, hyperopia, and astigmatism in which a laser is used to reshape the cornea to improve visual acuity, e.g., the clearness and sharpness of an image. The LASIK procedure involves both a surgical cutting and laser sculpting of the cornea. During LASIK, the eye is immobilized by application of a soft corneal suction ring. A flap in the outer cornea is then created using a blade or laser leaving a hinge on one end of the flap. The flap is then folded back to expose the stroma, or middle section of the cornea. A laser is then used to vaporize the corneal stroma to remove tissue to reshape the cornea to correct vision. After the stromal layer is reshaped, the flap is repositioned over the eye and remains in position by natural adhesion. Optimal visual acuity is usually achieved within about 24 hours following surgery.

The first step in the LASIK procedure is to slice the cornea from the side to produce a corneal flap that includes the epithelium and a portion of the stroma. The flap is cut using a device called a microkeratome. A part of the microkeratome flattens the cornea during the slice so as to create a corneal flap of uniform thickness. The slice is completed before a complete disk is created, which results in a corneal flap of uniform thickness with a hinge at one edge. The surgeon then rolls the flap back to expose an inner portion of the cornea. With the flap folded back, the surgeon performs the refractive correction on the inner portion of the cornea using a laser, such as an excimer laser. When the corneal sculpting is complete, the flap is repositioned into its original position and the procedure is complete. The eye has a natural suction facility that typically retains the flap in place when repositioned on the cornea.

FIG. 3B shows the portion of the eye 300 illustrated in FIG. 3A following LASIK. Following LASIK the optical region including center portion 311 of the stroma 305 has been sculpted as indicated by a thinner center thickness and flap 313 including incision 314 and the center portion of the cornea 303 including stroma 305 and epithelium 303 are repositioned and conform to the portion of the ablated stroma 305. The ablated stoma is characterized by an ablated profile 310P and bounded by unablated outer portions 312.

Although the LASIK surgical procedure is widely used, certain aspects of the procedure occasionally give rise to complications. For example, formation of the corneal flap is one aspect of the overall LASIK procedure which can give rise to complications. Specifically, the formation of the corneal flap can result in epithelial abrasions or other damage due to the microkeratome blade, and the cut or incision by means of the microkeratome blade can sometimes be unpredictable. Moreover, in some patients, corneal haze or edema subsequent to surgery and flap wrinkles or curled flap edges have been attributed to problems in forming the corneal flap with the microkeratome. The failure of the flap to reseal following surgery is also a significant complication because such failure creates a greater risk of infection and may adversely affect visual acuity.

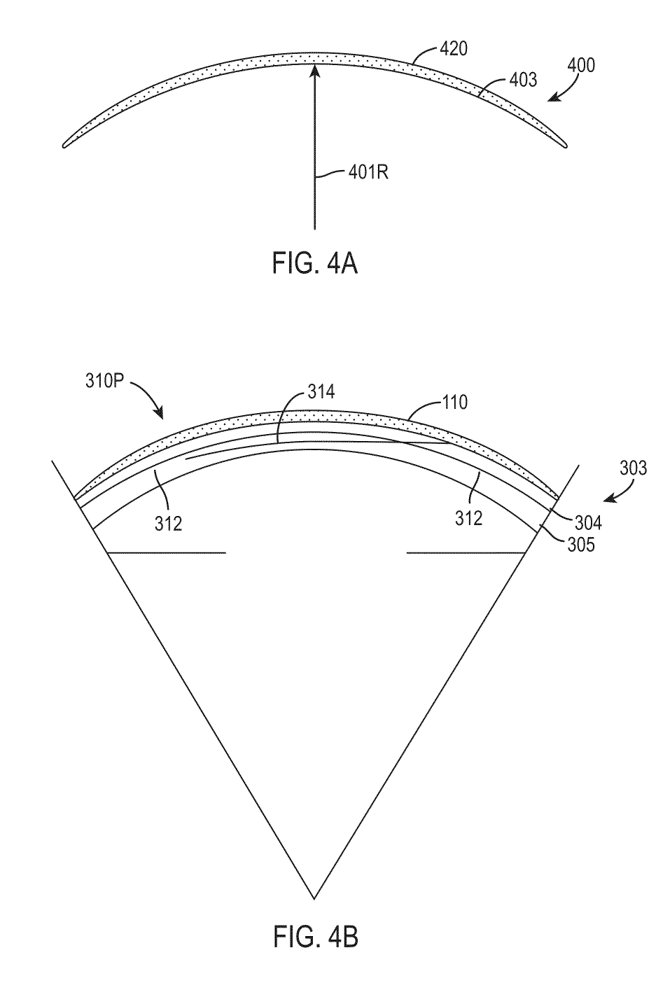

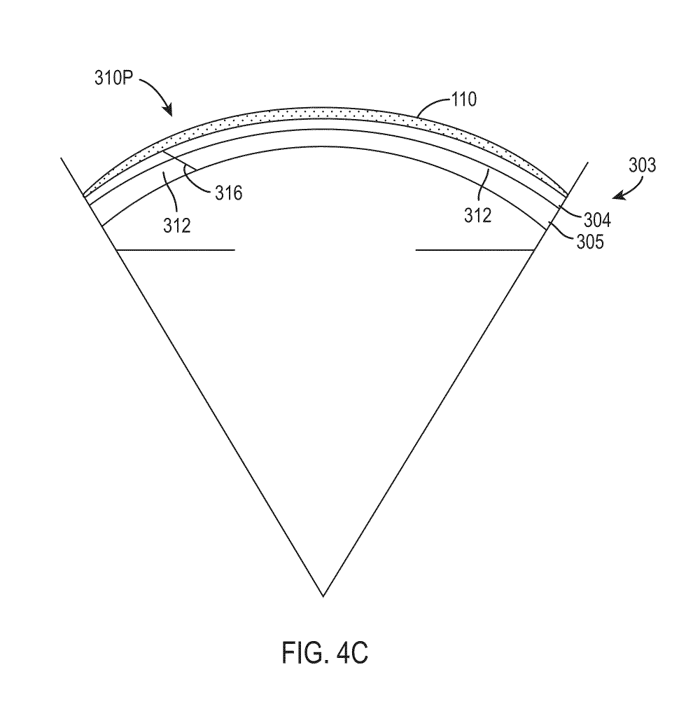

An optimal level of vision, e.g., 20/20 vision, is typically achieved within about 24 hour following LASIK surgery. Suboptimal vision can manifest as a myopic refractive error immediately following LASIK surgery, which gradually diminishes during the first 24 hours. This phenomena is shown in FIG. 5 and in FIG. 6. FIG. 5 shows the uncorrected visual acuity (UCVA) and best corrected visual acuity (BCVA) during the first 24 hours following LASIK surgery. Immediately following LASIK, a refractive correction of about 0.5D can be required to correct vision, which decreases to about 0.05D within the first about 4 hours. Thus, following LASIK, optimal vision is not achieved until about 24 hours post-surgery. In FIG. 6, the refractive change (spherical equivalent, SE) is shown following LASIK surgery. It is believed that the refractive error is caused by steepening of the corneal curvature due to edema and failure of the stromal flap to completely conform to the unablated and ablated stromal surfaces when repositioned on the cornea. Optical irregularities of the flap may in turn cause high order aberrations and/or the formation of an opaque bubble layer, which results in the BCVA not being 20/20 or better during the first few hours following surgery. The combination of flap irregularities and edema results in light scattering.

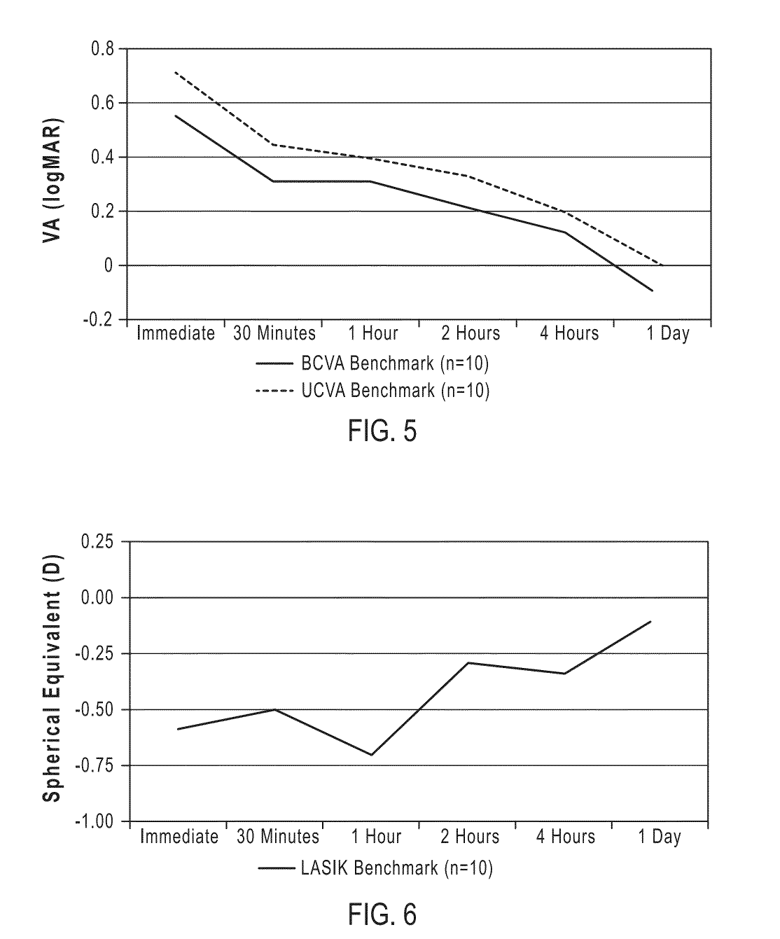

FIG. 7A and FIG. 7B illustrate the optical irregularities of the anterior surface of a cornea following LASIK as measured using a corneal topographer.

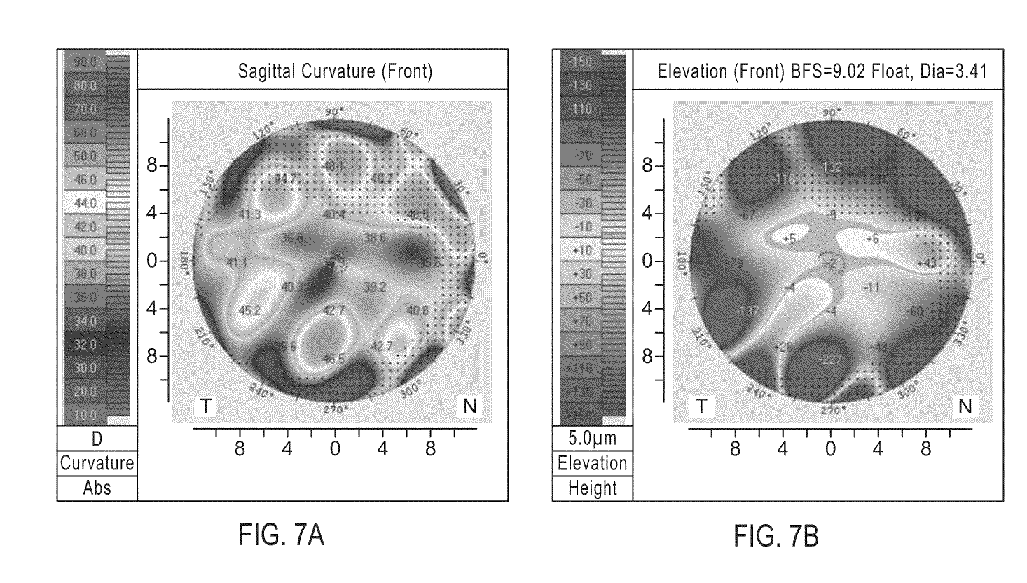

The effects of post-LASIK edema of corneal tissue is illustrated in FIG. 8A and FIG. 8B. FIG. 8A and FIG. 8B show photographs of cross-section of the optical region of the cornea including the stroma and overlapping epithelium before (FIG. 8A) and after (FIG. 8B) LASIK. A cross-sectional view of the optical region of the cornea prior to LASIK including stroma 801 and epithelium 802 is shown in FIG. 8A. The thickness of the stroma across the profile is 501 μm. LASIK surgery was then performed with a 120 μm interlase flap and the stroma was ablated to a depth of 42 μm across the section. FIG. 8B shows the same corneal cross-section showing ablated stroma 801, epithelium 802, and interlase flap interface 803 immediately following LASIK. As shown in FIG. 8B, edema causes the stroma to swell to a thickness from 572 μm to 591 μm across the same stromal section after the 42 μm ablation. The interface 83 between the LASIK flap and un-ablated stroma is also visible in the figure. The irregular corneal surface topography caused by the edema results in optical scattering, which manifests to a patient as halos around objects.

The combined effects of edema and optical defects result in suboptimal visual acuity following LASIK. Although vision improves dramatically during the first four hours following surgery and reaches an optimal post-operative state within about 24 hours for the majority of patients, it is desirable that vision be restored as soon as possible after a LASIK procedure to enable patients to function normally and to enhance psychological outcomes.

Coverings provided by the present disclosure may also be used as preventative devices. For example, coverings may be used to protect an eye from a potential injury such as injury due to physical trauma, protection from chemicals, protection from particulates, and protection from edema. As a preventative device, a covering can be applied to the eye prior to an anticipated exposure to a potential injury. When worn for protecting the eye from a potential injury, a covering can provide a physical barrier, a chemical barrier by virtue of the seal to the anterior surface of the eye, and/or may prevent or minimize edema caused by non-physical force such as blast pressure or by trauma to other parts of the body. In certain embodiments, protecting the eye form potential injury includes protected the eye from gases, vapors, dust, or smoke. In certain embodiments, protecting includes protected from edema.

When applied to an eye following ocular manipulation, coverings provided by the present disclosure improve visual acuity immediately following the surgical procedure, increase the rate at which optimal visual acuity is achieved, and reduce patient discomfort compared to recovery without wearing the covering.

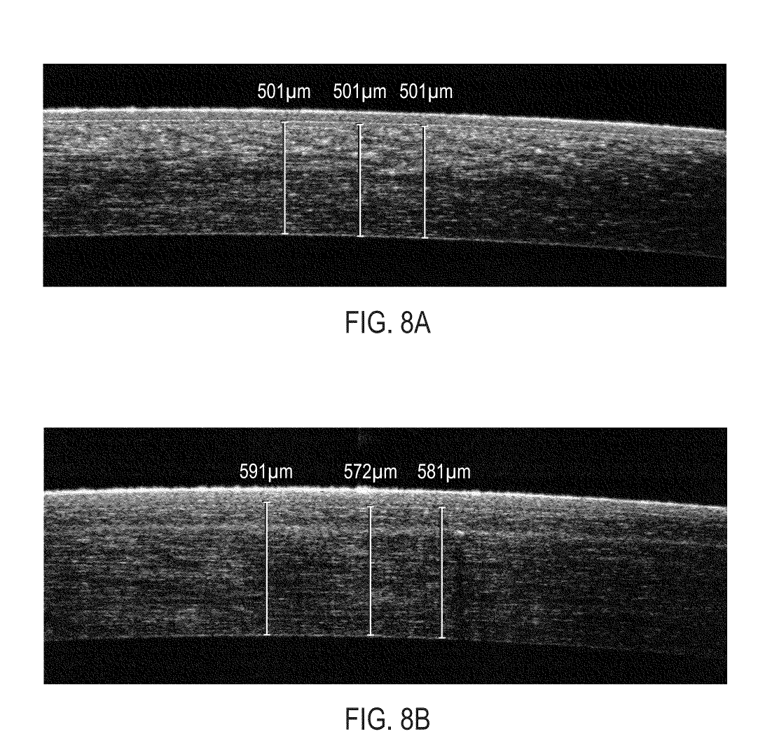

Certain embodiments provided by the present disclosure include coverings comprising an optical component and a coupling component, the optical component having a first rigidity, and the coupling component having a second rigidity, wherein the first rigidity is greater than the second rigidity. FIG. 9 shows covering 900, comprising optical component 901 and coupling component 902. Optical component 901 is also referred to herein as the inner portion and the coupling component 902 as the outer portion. The patient sees through the optical component or inner portion.

The inner portion is configured to optically conform to the cornea of the patient when applied to the eye. The inner portion may comprise one or more materials of the same or different thicknesses each material having the same or different modulus. In certain embodiments, the inner portion comprises a single material that is substantially homogeneous throughout the thickness. In certain embodiments, the inner portion comprises a first material that extends along the inner surface of the inner portion and contacts the outer surface of the cornea, and a second material anterior to the first material. In such embodiments, the first material can be relatively thin, have a lower modulus than the second material, and can be configured to provide comfort. The second material can have a modulus that is greater than that of the first material and can be configured to provide proper function of the covering and in certain embodiments, to correct vision.

The inner portion may or may not provide for vision correction. In certain embodiments, a covering may be used to treat an eye of a patient following an ocular therapy. The ocular therapy may be sufficient to correct vision and the covering is used to eliminate or minimize post-therapy complications and/or speed the time to optimal outcome. In such embodiments, the covering functions to heal the ocular tissue manipulated during the surgery and to improve visual acuity and other diagnostic criteria within at least the first few hours such as during the first at least 4 hours, to several days following the ocular therapy. In certain embodiments in which the inner portion does not provide vision correction, the inner portion can be characterized by a substantially uniform thickness and may be characterized by a refractive index that is substantially the same as the refractive index of the cornea. In certain embodiments, a substantially uniform thickness refers to a difference in thickness across the inner portion of less than 20 μm, less than 10 μm, less than 5 μm, and in certain embodiments, less than 1 μm. In other embodiments, the inner portion can be characterized by a shape having a spheric shape or an aspheric shape. A suitable shape can be selected determined on the degree and type of vision correction desired. Certain embodiments in which the inner portion is configured to correct refractive error, the inner portion may be characterized by a refractive index that is not substantially the same as the refractive index of the cornea.

The inner portion has an inner surface that optically conforms to the anterior surface of the cornea. In certain embodiments, the inner portion is characterized by at least one inner radius of curvature such as one radius of curvature, two radii of curvature, or more than two radii of curvature. In certain embodiments, the at least one inner radius of curvature comprises a radius of curvature corresponds to or is less than that associated with the optical power of the ablated stroma. In certain embodiments the inner portion comprises at least one inner radius of curvature that is equal to or flatter than the post-surgical profile of the cornea so that applying the covering comprises decreasing the at least one inner radius of curvature along the inner surface of the inner portion. In certain embodiments, the at least one inner radius of curvature is flatter than the post-ablation profile of the cornea by about 0D to about 3D.

In certain embodiments, the index of refraction of the inner portion is substantially the same as the index of refraction of the cornea. In embodiments in which the inner portion provides for vision correction, the refractive index of the inner portion may be different than that of the cornea such as greater than the refractive index of the cornea or less than the refractive index of the cornea.

In certain embodiments, the at least one inner radius of curvature comprises a radius of curvature corresponding to or less than the radius of curvature associated with the optical power of the cornea; and the method further comprises deforming the inner portion during the applying of the covering so that the upper surface of the inner portion optically conforms to the anterior surface of the cornea such that patient vision through the covering benefits from the ocular manipulation such that patient vision through the covering benefits from the therapy such that, within 4 hours of the therapy, patient discomfort associated with the therapy is mitigated and/or patient vision associated with the therapy is enhanced.

In certain embodiments, the at least one inner radius of curvature corresponds to or is flatter than the post-ablation profile of the cornea, so that applying the covering comprises decreasing the at least one inner radius of curvature along the inner surface of the inner portion of the covering.

In certain embodiments, the at least one inner radius of curvature is flatter than the post-ablation profile of the cornea by about 0D to about 3D; and the method further comprises deforming the inner portion during the applying of the covering so that the upper surface of the inner portion optically conforms to the anterior surface of the cornea such that patient vision through the covering benefits from the post-therapy profile and so as to modify tissue response of the cornea to the therapy such that, within 4 hours of the therapy, patient discomfort associated with the therapy is mitigated and/or patient vision associated with the therapy is enhanced.

In certain embodiments, the outer portion comprises at least one intermediate portion and a peripheral portion. The at least one intermediate portion is configured to at least partially conform to the cornea and the peripheral portion is configured to engage the eye so as to prevent movement of the inner portion. Both the at least one intermediate portion and the peripheral portion are configured to flex or deform when applied to an eye of the patient. Furthermore, the peripheral portion is configured to engage the epithelium, sclera, and or a combination of both to form a seal to the anterior surface of the cornea. The engagement of the peripheral portion prevents or minimizes motion of the covering when applied to the eye. The seal prevents or minimizes leakage of ocular fluids out of the eye and prevents or minimizes fluids such as tear fluid from entering the eye.

The inner surface of each of the at least one intermediate portion may independently be characterized by at least one radius of curvature. The radius of curvature defining each of the intermediate portions may have a focus at a point along the central axis of the covering (on-axis) or may have a focus off the central axis of the covering (off-axis). In certain embodiments, a covering may have from one to six intermediate portions, from two to six intermediate portions, or from four to six intermediate portions where each of the intermediate portions is characterized by a different radius of curvature. In certain embodiments, a covering comprises one intermediate portion, two intermediate portions, three intermediate portions, four intermediate portions, five intermediate portions, or six intermediate portions.

In certain embodiments, the peripheral portion may also comprise a lower surface characterized by at least one radius of curvature. The shape of the lower surface of the peripheral portion of the covering is configured to deform or flex to engage the outer portion of the eye along the epithelium, the sclera, or a combination thereof to resist movement of the inner portion. For use in optical therapies associated with incision of ocular tissue the peripheral portion and to a certain extent the at least one intermediate portion can be configured to engage the tissue anterior to the incision so as to hold the incised tissue against the cornea and/or other posterior ocular tissue and thereby promote healing.

In certain embodiments, the rigidity of the inner portion of the covering is greater than the rigidity of the outer portion. For example, in certain embodiments, a covering can have an inner rigidity from about 1.2E⁻⁶ Pa-m³ to about 3.1E⁻³ Pa-m³, from about 1E⁻⁵ Pa-m³ to about 1E⁻³ Pa-m³, and in certain embodiments, from about 1E⁻⁴ Pa-m³ to about 1E⁻³ Pa-m³.

In certain embodiments, a covering can have an outer rigidity from about 5.4E⁻⁹ Pa-m³ to about 1.5E⁻⁴ Pa-m³, from about 1E⁻⁸ Pa-m³ to about 1E⁻⁴ Pa-m³, from about 1E⁻⁷ Pa-m³ to about 1E⁻⁵ Pa-m³, and in certain embodiments, from about 1E⁻⁶ Pa-m³ to about 1E⁻⁵ Pa-m³.

The rigidity of a portion of the covering can be increased by increasing the thickness of a single material, using a material having a higher modulus for the same thickness, or by combining materials having different moduli and thicknesses.

The rigidity of a portion of a covering is approximated by the modulus of the material comprising the portion multiplied by the cube of the thickness. When a portion comprises more than one material, the rigidity can be approximated based on the average modulus of the portion multiplied by the thickness cubed of the portion. For example, a portion comprising a first material with a modulus of 20 MPa and a thickness of 90 μm and a second material with a modulus of 5 MPa and a thickness of 10 μm will have an average modulus of 18.5 MPa. The rigidity of the portion can then be approximated by multiplying the average modulus times the cube of the thickness, which for the present example is determined to be 18.5E⁻⁶ Pa-m³. Although these calculations can be based on approximations, a person skilled in the art can conduct simulations, for example finite element modeling simulations, so as to more accurately estimate relative rigidity and/or can measure pressures and deflection forces to determine rigidities of the various portions of the covering.

In certain embodiments, an inner portion of a covering is further characterized by an index of refraction that may correspond substantially to the index of refraction of the cornea, for example the index of refraction may be within a range from about 1.38 to about 1.43 so as to match the index of refraction of the cornea to within about +/−0.05. In certain embodiments, the inner portion and the outer portion are characterized by an index of refraction from about 1.38 to about 1.43 so as to match the index of refraction of the cornea to within about +/−0.05.

In certain embodiments, for example, where the covering provides vision correction, the inner portion may be characterized by an index of refraction that is different than the refractive index of the cornea.

Referring to FIG. 9, in certain embodiments, covering 900 has a diameter 910 from about 9 mm to about 16 mm, in certain embodiments, from about 10 mm to about 15 mm, and in certain embodiments, from about 12 mm to about 14 mm.

In certain embodiments, optical component 901 comprises a center thickness 905 from about 100 μm to about 500 μm, from about 200 μm to about 400 μm, from about 250 μm to about 350 μm, from about 250 μm to about 325 μm, and in certain embodiments, from about 250 μm to about 300 μm.

In certain embodiments, inner portion 901 comprises a first material having a first thickness 905 and a second material having a second thickness 906. In such embodiments, the second material may be disposed on the inner surface 911 of inner portion 901, e.g., the surface facing the cornea, and may be the same material as the material forming the outer portion. The second material may have a thickness 906 from about 5 μm to about 60 μm, from about 10 μm to about 50 μm, and in certain embodiments, from about 20 μm to about 40 μm. In such embodiments, where inner portion 901 comprises two materials, the total thickness of the optical component may be from about 100 μm to about 550 μm, from about 200 μm to about 450 μm, and in certain embodiments, from about 250 μm to about 350 μm. Furthermore, in certain embodiments, portion 901 may be characterized by a substantially uniform thickness as shown in FIG. 9.

In certain embodiments, inner portion 901 comprises an optically clear material having a modulus from about 10 MPa to about 70 MPa, from about 20 MPa to about 60 MPa, from about 20 MPa to about 50 MPa, and in certain embodiments from about 30 MPa to about 40 MPa. In certain embodiments, the inner portion comprises a material characterized by a modulus from about 20 MPa to about 30 MPa, from about 22 MPa to about 28 MPa and in certain embodiments about 25 MPa.

In certain embodiments, the inner portion of a covering comprises a single material having a modulus from about 1.2 MPa to about 25 MPa, a thickness from about 100 μm to about 500 μm, and a rigidity from about 1.2E⁻⁶ Pa-m³ to about 3.1E⁻³ Pa-m³. In certain embodiments, the outer portion of a covering comprises a single material having a modulus from about 0.2 MPa to about 1.4 MPa, a thickness from about 30 μm to about 500 μm (e.g., tapering from the thickness of the inner portion), and a rigidity from about 5.4E⁻⁹ Pa-m³ to about 1.5E⁻⁴ Pa-m³. In certain embodiments, the inner portion of a covering comprises a single material having a modulus from about 1.2 MPa to about 25 MPa, a thickness from about 100 μm to about 500 μm, and a rigidity from about 1.2E⁻⁶ Pa-m³ to about 3.1E⁻³ Pa-m³; and the outer portion of a covering comprises a single material having a modulus from about 0.2 MPa to about 1.4 MPa, a thickness from about 30 μm to about 500 μm (e.g., tapering from the thickness of the inner portion), and a rigidity from about 5.4E⁻⁹ Pa-m³ to about 1.5E⁻⁴ Pa-m³.

Referring to FIG. 9, inner portion 901 may be configured to correct vision or may not be configured to correct vision. For example, inner portion 901 may be characterized by a refractive index that is different than that of the cornea and/or may be spherically shaped, aspherically shaped, or may be another suitable shape that provides for vision correction. In such embodiments, the inner surface of the inner portion of a covering is configured to optically conform to the profile of the cornea of a patient's eye.

In certain embodiments, inner portion 901 comprises a material selected from silicone, silicone hydrogel, and a combination thereof. In certain embodiments, optical component 901 comprises silicone, in certain embodiments, silicone hydrogel, and in certain embodiments a combination of silicone and silicone hydrogel.

In certain embodiments, inner portion 901 comprises a center thickness from about 150 μm to about 500 μm, a diameter from about 3 mm to about 9 mm, a radius of curvature from about 7 mm to about 12 mm, and a modulus from about 20 MPa to about 50 MPa.

In certain embodiments, outer portion 902 extends from inner portion 901 to an outer periphery 904, where the thickness 907 at the juncture with inner portion 901 is the same as or similar to that of outer portion 902, and gradually tapers toward outer periphery 904, wherein the thickness of the coupling component at the periphery is from about 5 μm to about 60 μm, from about 10 μm to about 50 μm, and in certain embodiments, from about 20 μm to about 40 μm.

In certain embodiments, outer portion 902 comprises at least one radius of curvature. For example, in certain embodiments, outer portion 902 comprises a single radius of curvature, and in certain embodiments, outer portion 902 comprises more than one radius of curvature such as two, three, four, five, six, or more than six radii of curvature. The at least one radius of curvature can be, for example, from about 5 mm to about 15 mm, from about 6 mm to about 13 mm, from about 7 mm to about 12 mm, and in certain embodiments, from about 6 mm to about 10 mm. The one or more radius of curvature characterizing outer portion 902 is less than the radius of curvature of inner portion 901.

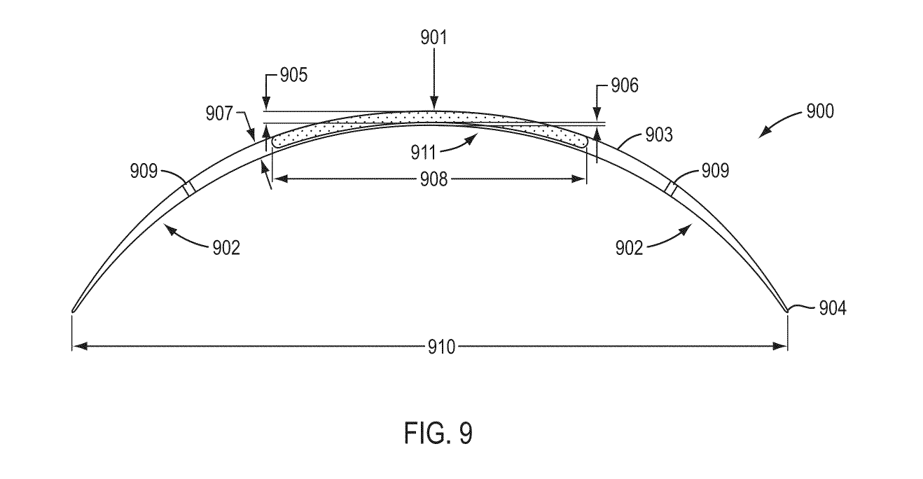

FIG. 10 shows a cross-section view of a covering according to certain embodiments of the present invention. The covering shown in FIG. 10 has a least a tri-curve profile including a central curvature, a mid-periphery curvature, and a peripheral curvature. The central curvature refers to the curvature of the inner portion of the covering spanning an approximately 3 mm diameter region in the center of the covering. The mid-periphery curvature refers to the curvature in a radial region about 5 mm from the center of the covering. The peripheral curvature refers to the curvature toward the edge of the covering. In certain embodiments, as shown for example in FIG. 10, the transition from the peripheral curvature region to other parts of the covering may not be smooth and may be characterized by an angle. FIG. 10 shows a centerline 1001 of coverings 1000 provided by the present disclosure, having a central region 1002 and mid-peripheral regions 1004 on either side of the central region 1002. In certain embodiments, the diameter 1003 of central region 1002 is from about 5 mm to about 7 mm, from about 5.5 mm to about 6.5 mm, and in certain embodiments is about 6 mm. In certain embodiments, the mid-peripheral regions 1004 extend form the edge diameter of center region 1002 to about 5 mm from centerline 1001. Accordingly, the diameter of the mid-peripheral region can be from about 7 mm to about 11 mm, from about 7 mm to about 10 mm, from about 6.5 mm to about 11 mm, from about 6.5 mm to about 10 mm, and in certain embodiments, from about 6 mm to about 10 mm. In certain embodiments, the peripheral diameter 1007 of a covering can be from about 11 mm to about 16 mm, from about 12 mm to about 15 mm, and in certain embodiments, about 14 mm. As referred to herein, the outer portion comprises the mid-peripheral regions, which are also referred to as intermediate portions, and the peripheral portion.

Referring to FIG. 9, in certain embodiments, outer portion 902 comprises a material having a modulus from about 0.05 MPa to about 4 MPa, from about 0.1 MPa to about 3 MPa, from about 0.1 MPa to about 2 MPa, and in certain embodiment from about 0.2 MPa to about 1.5 MPa. In certain embodiments, the outer portion comprises a material characterized by a modulus from about 0.9 MPa to about 1.5 MPa, from about 1 MPa to about 1.4 MPa, and in certain embodiments, about 1.2 MPa. In certain embodiments, a covering comprises an inner portion characterized by a diameter of about 6.2 mm, a thickness of about 270 microns, and formed from a material such as a silicone polymer or silicone hydrogel characterized by a modulus of about 25 MPa, and an outer portion formed from a material such as a silicone polymer or silicone hydrogel characterized by a modulus of about 1.2 MPa, and wherein the covering has a diameter of about 14 mm.

Referring to FIG. 9, in certain embodiments, outer portion 902 comprises a material selected from silicone, silicone hydrogel, and a combination thereof. In certain embodiments, coupling component comprises silicone, in certain embodiments, silicone hydrogel, and in certain embodiments a combination of silicone and silicone hydrogel.

In certain embodiments, the material forming a covering including both the inner and outer portions have low water content and is characterized by low water or ion permeability. In certain embodiments, the water content is less than about 5%, less than about 4%, and in certain embodiments, less than about 3%. In certain embodiments, the material forming a covering has a water content less than about 1%, less than about 0.6%, and in certain embodiments, less than about 0.3%. In certain embodiments, the material less than about 0.4×10⁻⁶ cm²/sec, less than about 0.2×10⁻⁶ cm²/sec, and in certain embodiments, less than about 0.1×10⁻⁶ cm²/sec.

In certain embodiments, outer portion 902 does not include fenestrations 909 as shown in FIG. 9. In other embodiments, outer portion 902 may comprise fenestrations. For example, for use in methods of treatment in which it is desirable that a covering seal the anterior surface of the cornea, a covering does not include fenestrations.

In certain embodiments, outer portion 902 may comprise a thickness tapering from the thickness of inner portion 901 to a thickness of about 30 μm at the periphery 904 of the coupling component; a plurality or radius of curvature from about 7 mm to about 12 mm; and comprises a material having a modulus from about 0.1 MPa to about 2 MPa. In embodiments in which outer portion 902 comprises a plurality of radii of curvatures 912, the radius of curvature decreases from the optical component toward the periphery.

A covering, including inner portion 901 and outer portion 902, can be configured to provide a seal to a tissue of an eye such as an epithelium to thereby resist movement of the optical component on an eye and to prevent fluid such as tear fluid from getting under the covering, thereby enhancing edema recovery.

As disclosed herein, the inner portion comprises an inner surface characterized by at least one curvature, and the outer portion comprising an inner surface characterized by at least one of curvature. Furthermore, in certain embodiments, the outer portion comprises a peripheral portion characterized by a peripheral curvature, and at least one intermediate portion, each of the at least one intermediate portion independently characterized by an intermediate curvature. Each of the curvatures may be the same, each of the curvatures may be different, and in certain embodiments, at least some of the curvatures are the same. Furthermore, each of the curvatures may be spheric or aspheric. A spheric curvature can be characterized by a radius of curvature. An aspheric curvature encompasses a curvature that is not spheric.

In certain embodiments, the inner surface of a covering is characterized by at least one spheric curvature. In certain embodiments, the inner surface of a covering is characterized by at least one aspheric curvature. In certain embodiments, the inner surface of a covering is characterized by a combination of spheric curvatures and aspheric curvatures.

In certain embodiments, a radius of curvature may have a focus along an extension of the central axis of the covering. In certain embodiments, a radius of curvature may have a focus that is not located along an extension of the central axis of the covering. The extension of the central axis of the covering refers to a line segment extending from the center of the covering and projecting perpendicular to a plane defined by the peripheral edge of the covering.

In certain embodiments, the inner portion comprises an inner surface having a shape characterized by a single radius of curvature, by two radii of curvature, and in certain embodiments by three radii of curvature.

In certain embodiments, the outer portion of the covering comprises an inner surface characterized by a single radius of curvature, by two radii of curvature, by three radii of curvature, by four radii of curvature, by five radii of curvature, by six radii of curvature, or by more than six radii of curvature, such as from 8 to 10 radii of curvature or more. Thus, in certain embodiments the outer portion comprises an inner surface characterized from 1 to 6 radii of curvature, from 2 to 6 radii of curvature, and in certain embodiments, from 4 to 6 radii of curvature.

In general, the inner surface of the covering is configured to substantially conform to the anterior surface of the cornea. In certain embodiments, substantially conforming to the anterior surface of the cornea means that the inner portion of the covering and at least a portion of the outer portion of the covering physically contact the epithelium and/or stroma when the covering is applied to an eye of the patient. In certain embodiments in which the outer portion comprises a peripheral portion and at least one intermediate portion, the peripheral portion contacts the epithelium, sclera, or a combination thereof, and one or more of the intermediate portions may or may not contact the epithelium when the covering is applied to an eye of a patient.

Coverings provided by the present disclosure are configured to substantially conform to the anterior surface of the cornea when applied to the eye of the patient. A cornea, and in particular a post-therapy cornea, can be characterized by a topography that is directly and/or indirectly related to the therapy. For example, the corneal topography can be related to sculpting of the cornea, inlays or onlays, intraocular lenses or corneal transplants, as well as incision associated with the various surgeries. Indirectly, the surface of the cornea may be affected by irregularities associated with disturbed tissue and edema. A covering is configured such that when applied to the cornea the ocular tissue is compressed against the inner surface of the covering to substantially smooth the anterior surface of the cornea. Depending on the type of ocular therapy and the type of ocular defects, if any, the entire anterior surface of the cornea may not physically conform to the inner surface of the covering. For example, as disclosed herein, a covering is configured to bridge defects of the anterior corneal surface. Nevertheless, it should be appreciated that one of the benefits of a covering is to provide a smooth optical surface and therefore it is the anterior surface of the cornea that effectively substantially conforms to or physically accommodates to the smooth optical surface of the covering. When applied to the eye, the inner surface of a covering and the anterior surface can be said to at least partially conform. In certain embodiments, such as when there are no significant defects on the anterior corneal surface, a covering and the corneal surface may physically conform across the anterior surface of the cornea. In certain embodiments in which there are significant defects on the anterior corneal surface, a covering the corneal surface may partially conform such that in the region of the defect the covering and the cornea do not physically conform. At least partially conforms encompasses fully conforms, substantially conforms, and partially conforms. In certain embodiments in which the inner portion bridges epithelial and/or stromal defects, a covering such as the inner portion of the covering can be said to optically conform to the optical region of the cornea.

Coverings provided by the present disclosure may have different cross-sectional shapes. For example, in certain embodiments, the inner portion may have a substantially uniform thickness, and the cross-sectional thickness of the covering may taper toward the periphery of the covering.

In certain embodiments, the inner portion is not intended to provide for vision correction and in such cases, the inner portion has a substantially uniform thickness. Such embodiments are suitable, for example, when ocular therapy such as any of the surgical procedures disclosed herein provides adequate vision correction and additional correction is not necessary.

In certain embodiments, the inner portion may be characterized by a shape that provides vision correction. For example the inner portion may be characterized by a spheric or aspheric shape. In such embodiments, the outer surface of the inner portion may be characterized by a spheric or an aspheric profile.

In certain embodiments, the inner portion comprises a different material than the outer portion. In certain embodiments, the inner portion and the outer portion comprise the same material. In embodiments in which the inner portion and the outer portion comprise the same material, the different moduli may be realized by the detailed chemistry of the polymer used, such as characterized by different crosslinking densities.

In certain embodiments, the inner portion of a covering and the outer portion of a covering comprise a first material characterized by a first modulus and extending along a lower surface of the covering; and the inner portion comprises a second material characterized by a second modulus disposed anteriorly to the first material, the second modulus being greater than the first modulus. In such embodiments, the first material is a thin layer that is configured to promote comfort of the covering when applied to the cornea by cushioning between the anterior surface of the cornea and the layer of the first material. The second material is configured to promote a beneficial optical shape of an anterior surface of the applied covering over the eye.

The index of refraction of one or more layers of a covering may correspond substantially to the index of refraction of the cornea.

A covering may comprise one or more of many optically clear materials, for example synthetic materials or natural material such collagen-based materials, and combinations thereof, such as described in U.S. Publication No. U.S. 2010-0036488. For example, a covering may comprise a naturally occurring material, such as collagen based material. Alternatively or in combination, a covering material may comprise a known synthetic material, for example hydroxyethyl methacrylate (HEMA) hydrogel, hydrogel, silicone, for example hydrated silicone and derivatives thereof. For example the optically clear material may comprise one or more of silicone, silicone hydrogel, silicone comprising resin, silicone comprising silicate, acrylate, collagen, or a combination of any of the foregoing. The cured silicone may comprise silicone that is two-part, heat-curable and RTV (room temperature vulcanized). For example, polydimethyl siloxane such as NuSil, or poly(dimethyl) (diphenyl) siloxane may be used to mold the covering, for example with less than 10% water content so as to increase oxygen diffusion through the covering. A covering may comprise perfluoropolyethers or fluorofocal. The material may comprise, for example, silicone elastomer having optically clear silicate disposed therein and a water content of no more than about 10%, for example no more than about 5%, or no more than about 1%, such that the covering has a very high Dk exceeding 150 and in certain embodiments exceeding 300, and the silicone lens comprising silicate can be treated to provide a wettable surface. A covering may comprise hydrogel, for example silicone hydrogel, and can be formed with a water content within a range from about 5% to about 35% and a modulus within a range or a combination of ranges from about 0.1 MPa to about 40 MPa, such that the covering conforms at least partially to the anterior surface of the cornea. In certain embodiments, coverings provided by the present disclosure do not contain water and provide a barrier for the flow of fluid across the covering. For example, when applied to the cornea, coverings minimize or prevent the flow of fluid from the cornea and the flow of fluid such as tea fluid from the outer surface of the covering to the cornea. The coverings provide a fluid seal and the material or materials forming a covering are selected to minimize or prevent moisture transport across the covering thickness.

In certain embodiments, coverings provided by the present disclosure are characterized by a high oxygen permeability (Dk, cm²·mL O₂/sec·mL·mm Hg) such as from 100 to 500, from 200 to 500, from 250 to 450, from 300 to 400, and in certain embodiments, about 350. In certain embodiments, coverings provided by the present disclosure are characterized by a high oxygen permeability (Dk) such as at least about 250, at least about 300, at least about 350, and in certain embodiments, at least about 400.

A covering may comprise silicone or silicone hydrogel having a low ionoporosity such that a covering seals to the cornea. For example, a covering may comprise silicone hydrogel comprising a low ion permeability, and the range of water can be from about 5% to about 35%, such that the Dk is 100 or more. In certain embodiments, the low ion permeability may comprise an Ionoton Ion Permeability Coefficient of no more than about 0.25×10⁻³ cm²/sec so as to seal the cornea, for example no more than about 0.08×10⁻³ cm²/sec. In certain embodiments, the low ion permeability comprises an Ionoton Ion Permeability Coefficient of no more than about 2.6×10⁻⁶ mm²/min to seal the cornea, for example no more than about 1.5×10⁻⁶ mm²/min.

A covering may comprise a wettable surface coating disposed on at least the upper side of the covering, such that the tear film of the patient is smooth over the covering and the patient can see. The wettable surface coating may comprise a lubricious coating for patient comfort, for example to lubricate the eye when the patient blinks. The wettable coating may comprise a contact angle no more than about 80 degrees. For example the coating may comprise a contact angle no more than about 70 degrees and the contact angle can be within a range from about 55 degrees to 65 degrees to provide a surface with a smooth tear layer for vision. For example, the wettable coating can be disposed both an upper surface and a lower surface of the covering. The upper surface may comprise the wettable coating extending over at least the inner portion of the covering.

A wettable coating may comprise one or more of many materials. For example, a wettable coating may comprise polyethylene glycol (PEG), and the PEG coating can be disposed on Parylene™. Alternatively, a wettable coating may comprise a plasma coating, and a plasma coating may comprise a luminous chemical vapor deposition (LCVD) film. For example, in certain embodiments a plasma coating comprises at least one of a hydrocarbon, for example CH₄, O₂ or fluorine containing hydrocarbon, for example CF₄ coating. Alternatively or in combination, a wettable coating may comprise a PEG) coating or 2-hydroxyethylmethacrylate (HEMA). For example, a wettable coating may comprise HEMA disposed on a Parylene™ coating, or a wettable coating may comprise N-vinylpyrrolidone (NVP) disposed on a Parylene™ coating.

Appropriate covering dimensions can be determined in many ways, for example, with topography measurements of the cornea and sclera. Corneal and scleral topography can be measured with many instruments, such as with the Orbscan™ topography system commercially available from Bausch and Lomb, the Pentacam™ Scheimpflug camera system commercially available from Oculus, and commercially available optical coherence tomography (OCT). The ablation profile can be combined with the topography to determine the shape of the eye.

The dimensions of a covering can be sized to one or more of the cornea and sclera based on tolerances that may be determined clinically.

To speed visual recovery following ocular therapy, methods provided by the present disclosure comprise providing a covering to the eye of a patient immediately following ocular therapy such as within the first few minutes following ocular therapy. Benefits may also be realized following application of a covering within the first few hours or days following ocular therapy. A covering may be worn for a few hours following application, for a few days, or until recovery from the ocular therapy is complete or nearly complete.

A covering may be applied to the eye of a patient at any appropriate time following ocular therapy, such as immediately after the ocular procedure is completed. A covering may be applied by approximately centering the covering on the cornea of a patient's eye and providing a slight pressure against the covering to flex the covering against the cornea such that the covering is retained against the cornea. The moduli of the materials and the shape of the inner surface of the covering from the center to the periphery of a covering are selected to enable the inner surface of the covering to optically conform to the anterior surface of the cornea, to retain the covering against the cornea during normal wear, to seal the anterior surface of the cornea, to immediately improve visual acuity, to speed the recovery of optimal visual acuity, and/or to enhance patient comfort.

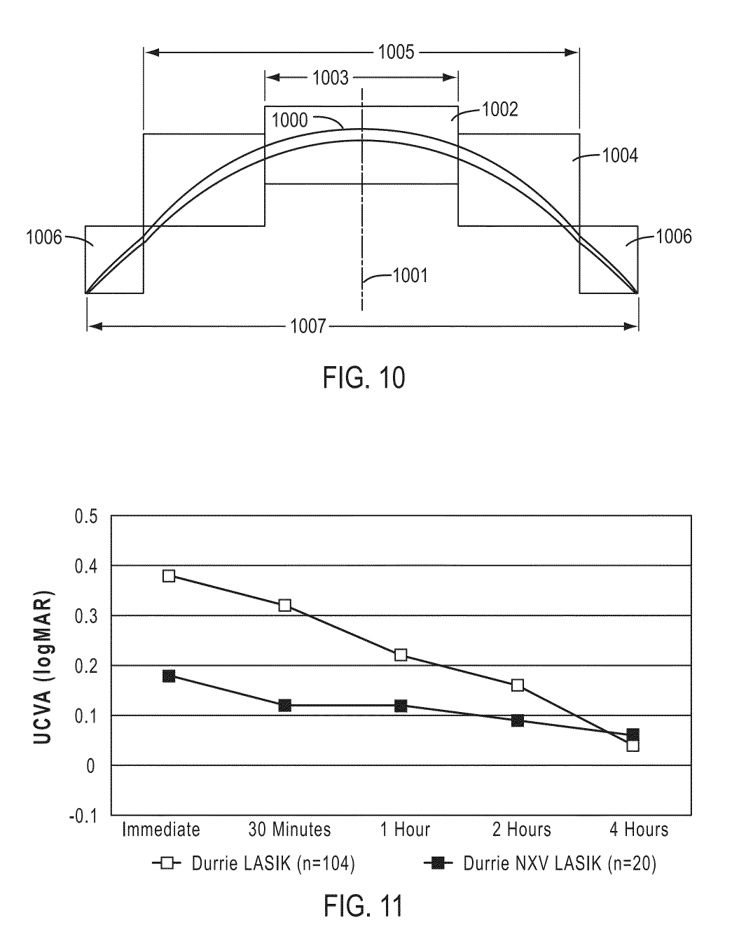

Improvement in visual recovery provided by methods of the present disclosure is shown in FIG. 11. In FIG. 11, the monocular UCVA is shown at various times following LASIK surgery. Without a covering applied to the eye, the UCVA is about 0.4 log MAR corresponding to about 20/50 vision immediately following LASIK and the UCVA gradually decreases toward 0 log MAR corresponding to 20/20 vision within the first 4 hours. In comparison, when a covering is applied following LASIK, the initial UCVA is about 0.2 log MAR corresponding to 20/32 vision, which gradually improves toward 20/20 vision over the next 4 hours. The ability of the covering to improve vision immediately following LASIK can be attributed to the ability of the covering to provide an optically smooth corneal surface. For example, when the LASIK flap is repositioned over the ablated stroma, the flap may not reposition evenly over the surface of the ablated stroma and thereby create irregularities. The mechanics of the applied covering serves to smooth the LASIK flap against the stroma. Thus, when applied, a covering has an immediate effect in improving visual acuity as the result of the optical smoothing. In terms of the mechanics, it is believed that a covering provided by the present disclosure imposes a negative pressure or suction against the anterior surface of the cornea sufficient to bring ocular tissue toward the inner surface of the covering, which is held in place by engagement with the peripheral portion with the eye, and thereby smooth the ocular tissue against the inner surface of the covering.

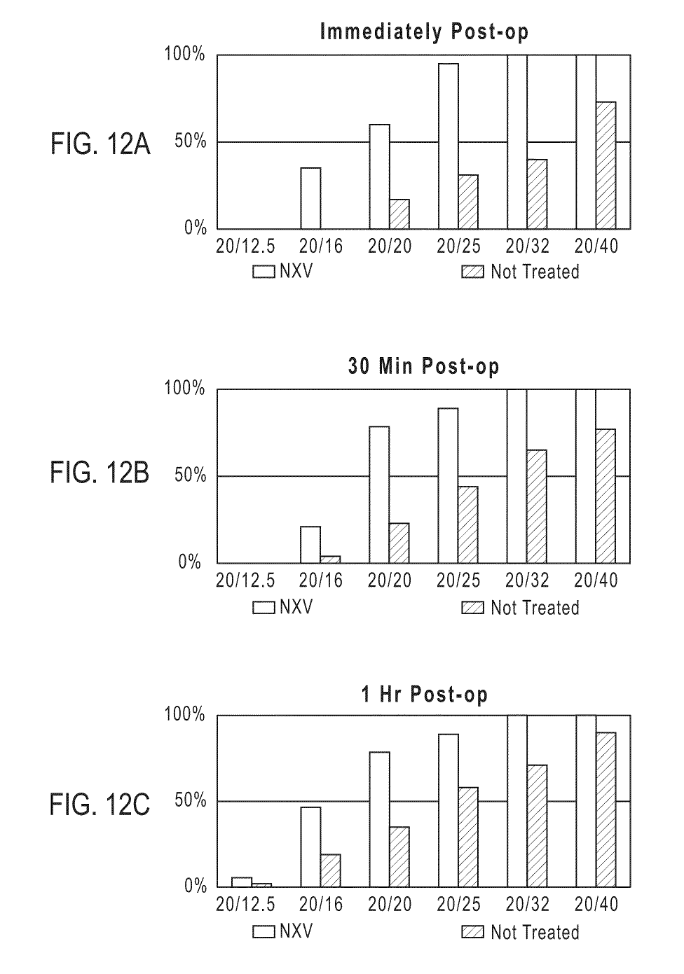

The ability of a covering to decrease the time of visual recovery compared to the time of visual recovery without the covering was confirmed by measuring the binocular visual acuity in a population of patients following LASIK. FIGS. 12A-12F show histograms representing binocular visual acuities for a population of patients (n=20) with and without a covering provided by the present disclosure applied to an eye following LASIK. Each histogram represents the percent of the population of patients that reported the indicated binocular visual acuity or better. For example, as shown in FIG. 12A, immediately, i.e., within the first 5 minutes, following LASIK, about 60% of patients wearing a covering provided by the present disclosure reported 20/20 vision, compared to only about 20% of patients not wearing a covering. Similar improvements in vision for the population of patients wearing a covering is observed at 30 minutes (FIG. 12B), 1 hour (FIG. 12C), 2 hours (FIG. 12D), 4 hours (FIG. 12E), and up to 1 day (FIG. 12F) following surgery. These results also demonstrate that whether or not a covering is applied following LASIK, binocular visual acuity gradually improves during the recovery period. However, for those patients wearing a covering, binocular visual acuity is restored much more rapidly compared to LASIK patients not wearing a covering.

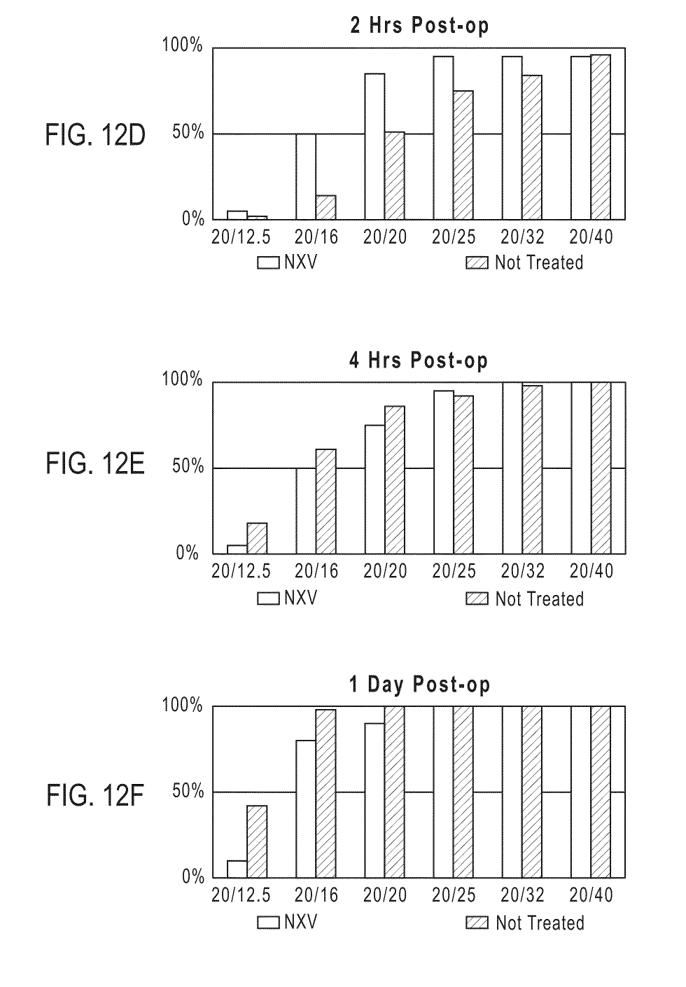

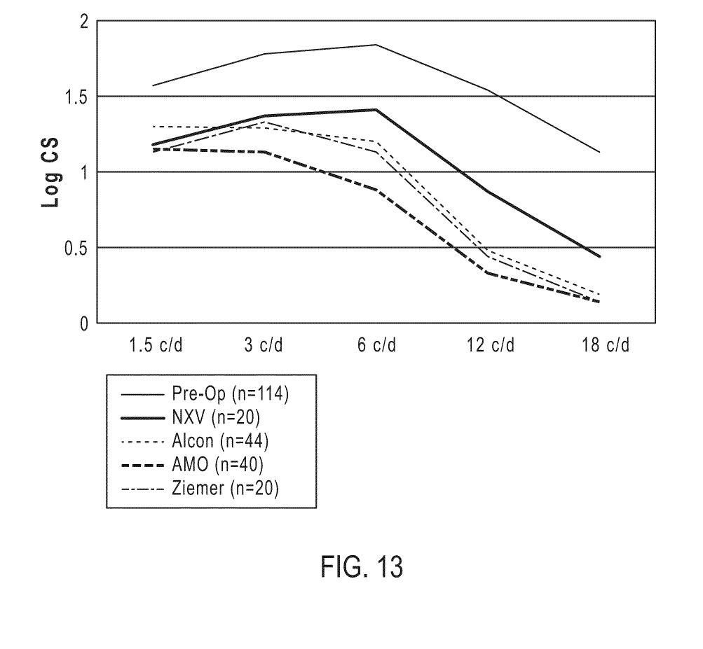

Coverings provided by the present disclosure also improve contrast sensitivity following LASIK surgery. Contrast sensitivity is a measure of the ability of the eye to discern between luminances of different levels in a static image. Contrast sensitivity can depend, for example, on the spatial frequency of an image. The improvement in contrast sensitivity using a covering provided by the present disclosure is provided in FIG. 13. FIG. 13 shows the monocular mesopic contrast sensitivity (log CS) as a function of the spatial frequency. The uppermost curve presents the frequency-dependent contrast sensitivity of a population of patients prior to LASIK. Following LASIK surgery using an Alcon, AMO, or Ziemer LASIK platforms, the contrast sensitivity, and in particular the high frequency contrast sensitivity, is decreased. Comparing the pre-operation contrast sensitivity with the contrast sensitivity following any of the surgical procedures noted, post-surgical edema is shown to cause an even depression of contrast sensitivity at all spatial frequencies. The additional decreased high frequency contrast sensitivity (from 12 c/d to 18 c/d) is attributed to refractive error and/or higher order aberrations. As shown in FIG. 13, when a covering provided by the present disclosure is applied to an eye following LASIK surgery, although there is modest if any improvement in image quality at low spatial frequencies (1.5 c/d to 6 c/d), the high spatial frequency (6 c/d to 18 c/d) contrast sensitivity is improved thereby indicating that a covering provides a sharper retinal image quality. These results further confirm that coverings provided by the present disclosure predominantly function to correct refractive error and/or higher order optical aberrations rather than to enhance vision by affecting post-LASIK edema immediately (within the first 5 minutes) following surgery. FIG. 14 shows the relationship between contrast sensitivity and visual acuity and the typical contrast sensitivity for normal vision (contrast sensitivity curve).

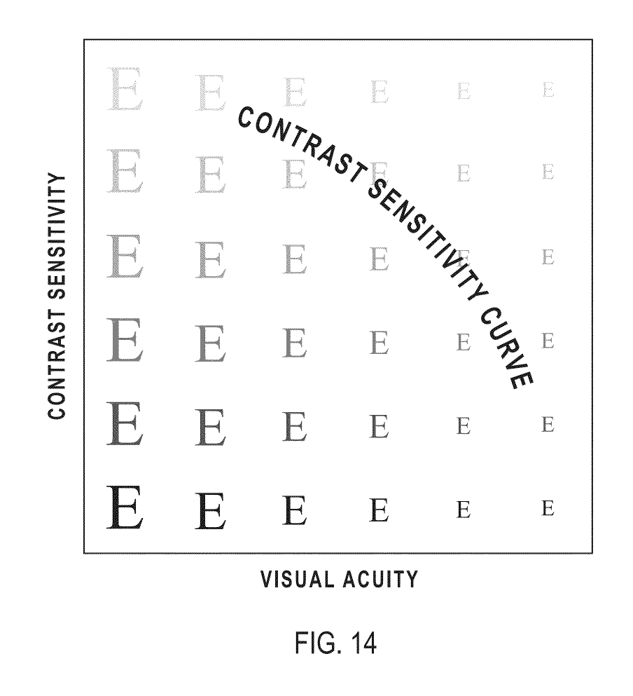

Nevertheless, coverings provided by the present disclosure also resolve edema over time resulting in reduced scattering and veiling glare and thereby restore vision faster. As shown in FIG. 15, post-operative edema was measured with and without the covering. Thirty-minutes following surgery, with the covering in place, edema caused by the LASIK procedure was reduced from about 100 μm to about 60 μm. The improvement in edema was observed throughout the 24-hour recovery period.

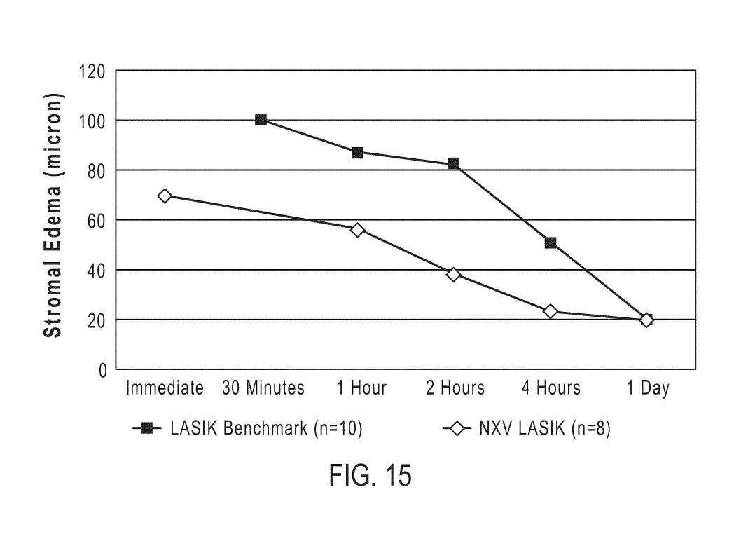

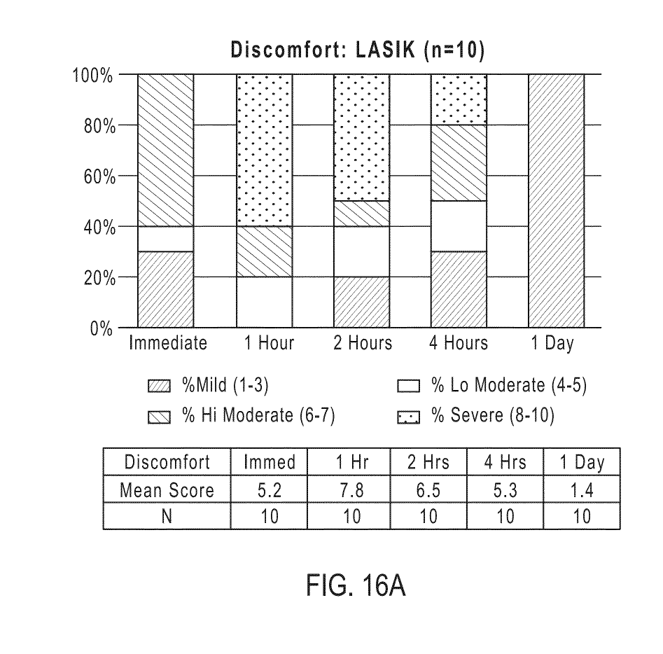

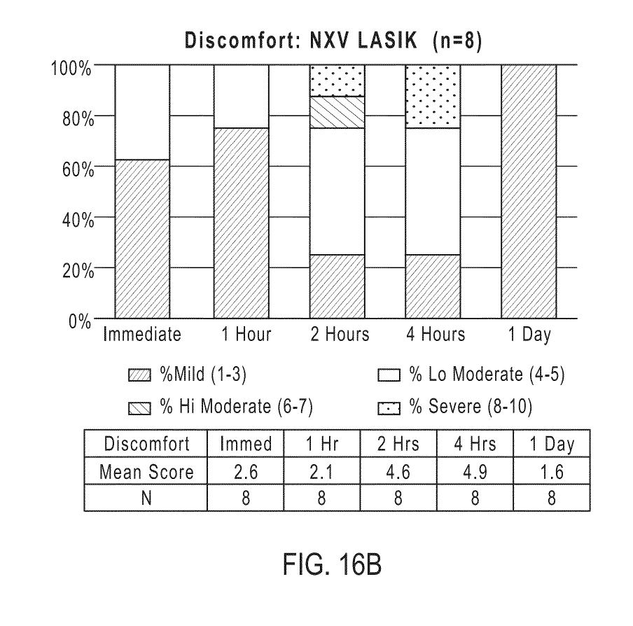

Coverings provided by the present disclosure also reduce post-LASIK discomfort as reflected in reduced overall discomfort, reduced outdoor photophobia, and reduced burning sensation. Following LASIK surgery, the majority, e.g., greater than 50%, of patients report light sensitivity (photophobia) and a burning sensation during the first 4 hours. In one study, wearing a covering provided by the present disclosure, 85% of patients (n=20 patients) reported no discomfort symptoms. Further details of studies measuring post-LASIK discomfort are summarized in FIG. 16A, FIG. 16B, FIG. 17A, FIG. 17B, FIG. 18A, and FIG. 18B.

Histograms summarizing the percent of patients reporting various degrees of overall discomfort following LASIK surgery are provided in FIG. 16A and FIG. 16B. As shown in FIG. 16A, without a covering, as reflected in the histograms, the mean overall discomfort score was 5.2 immediately following surgery, 7.8 at one hour following surgery, and 6.5 at two hours following surgery. In comparison, as shown in FIG. 16B, when a covering was worn, the mean overall discomfort score was 2.6 immediately following surgery, 2.1 at one hour following surgery, and 4.6 at two hours following surgery. The differences in the mean overall discomfort scores immediately following surgery, at one hour following surgery, and at two hours following surgery are statistically significant (α<0.01). Thus, application of a covering to an eye improves the overall discomfort during at least the first two hours following LASIK surgery.

Similar improvements are observed for outdoor photophobia. FIG. 17A and FIG. 17B show histograms of outdoor photophobia following LASIK surgery. For patients not wearing a covering, the mean outdoor photophobia was 5.3 immediately following LASIK surgery, 7.7 at one hour following surgery, and 7.8 at two hours following surgery. As shown in FIG. 17B, when a covering is applied to the eye following LASIK surgery, the corresponding mean outdoor photophobia scores are 2.6 immediately following surgery, 2.2 at one hour following surgery, and 4.4 at two hours following surgery. The differences in the mean outdoor photophobia scores immediately following surgery, at one hour following surgery, and at two hours following surgery are statistically significant (α<0.01). Thus, the results presented in FIG. 17A and FIG. 17B demonstrate that application of a covering to an eye improves the outdoor photophobia during at least the first two hours following LASIK surgery.

Coverings can also reduce the burning sensation during the first few hours following LASIK. As shown in FIG. 18A, for patients not wearing a covering, the mean burning sensation score was determined to be 5.6 immediately following surgery, 6.2 at one hour following surgery, and 9.0 at two hours following surgery. In comparison, as shown in FIG. 18B, when a covering is applied to the eye immediately following LASIK surgery, the mean burning sensation score was determined to be 3.9 immediately following surgery, 3.0 one hour following surgery, and 6.6 at two hours following surgery. The differences in the mean burning sensation scores one hour following surgery and two hours following surgery are statistically significant (α<0.01). Thus, coverings provided by the present disclosure significantly improve discomfort associated with a burning sensation at least within the first two hours following LASIK surgery.

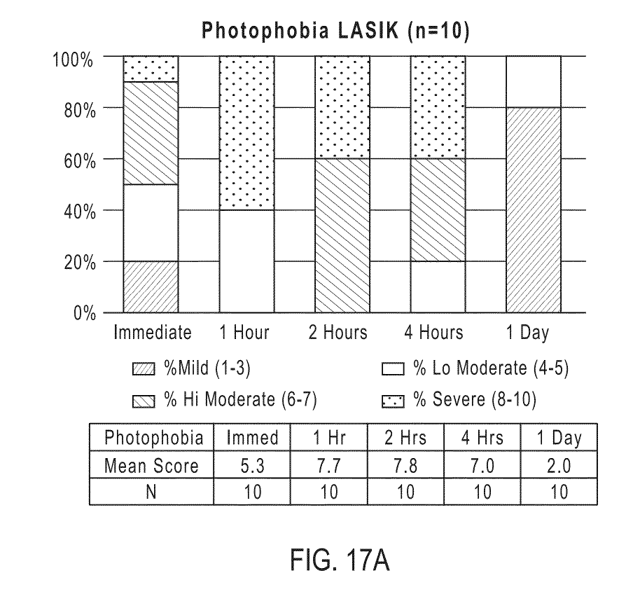

The ability of coverings to improve functional measures of visual recovery following LASIK surgery was also assessed. FIG. 19 shows the ability of a patient to text using a mobile telecommunications device such as a mobile phone at various times following LASIK surgery. To assess texting ability, patients were encouraged to text and to report their ability to text at each time. As is shown in FIG. 19, immediately following LASIK surgery, most patients were comfortable texting, however, measurably more patients wearing a covering were texting immediately following surgery, at 30 minutes following surgery, and at one hour following surgery, compared to those patients not wearing a covering.

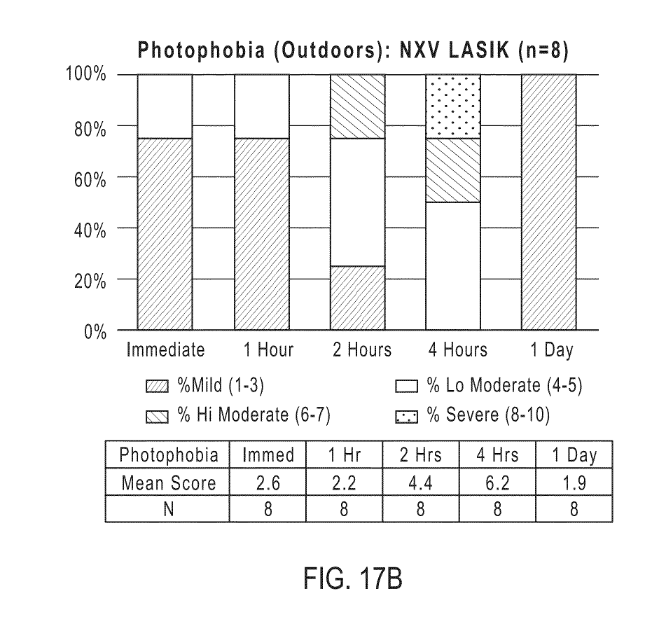

A similar improvement in the rate of recovery in a patient's perceived ability to drive following LASIK surgery when wearing a covering was also observed. FIG. 20 shows the percent of patients who felt comfortable driving at various times following LASIK surgery. Overall, patients who were wearing a covering felt more comfortable driving during the first 4 hours following LASIK surgery than did patients who did not wear a covering.

Fitting a covering to a patient's eye involves, at least in part, selecting a covering that at least partially restores vision following optical therapy, increases the rate at which optimal visual acuity is achieved, and improves patient comfort. In general, the parameters associated with the central 3 mm region of a covering determine properties associated with vision. In general, the parameters associated with the mid-peripheral 5 mm region, e.g., the intermediate portion, of a covering correlates with patient comfort. Furthermore, in generally there is little or no interaction between the fit of the 3 mm and the 5 mm regions. In certain embodiments, recommended fitting ranges for the 3 mm central region is from −1.0D to −2.5D than the corresponding central region of the cornea; and for the 5 mm mid-peripheral region is from about −1.0D to about −2.5D than the corresponding region of the cornea. In certain embodiments, recommended fitting ranges for the 3 mm central region is from −2.0D to −3.0D than the corresponding central region of the cornea; and for the 5 mm mid-peripheral region is from about −0.0D to about −1.5D than the corresponding region of the cornea.

Because a range of covering curvatures can be fit to a range of corneal curvatures in treating post-therapy vision, relatively few covering choices need be manufactured thereby reducing costs associated with stocking and facilitating the fitting procedure.

The fitting strategy for selecting a covering shape for a patients eye involves, at least in part, determining the curvature of the cornea of the eye of the patient, and selecting a covering that has a center curvature that is less than, e.g., flatter than, the curvature of the cornea. In certain embodiments, the curvature of the selected covering is from −1.0D to −2.0D than the curvature of the cornea, from −1.0D to −2.5D than the curvature of the cornea, from −1.0D to −3.0D than the curvature of the cornea, and in certain embodiments, from 0D to −3.0D than the curvature of the cornea. In certain embodiments, the curvature of the selected covering is from −1.5D to −2.5D than the curvature of the cornea, from −1.5D to −3.0D than the curvature of the cornea, and in certain embodiments, from −1.5D to −3.5D than the curvature of the cornea.

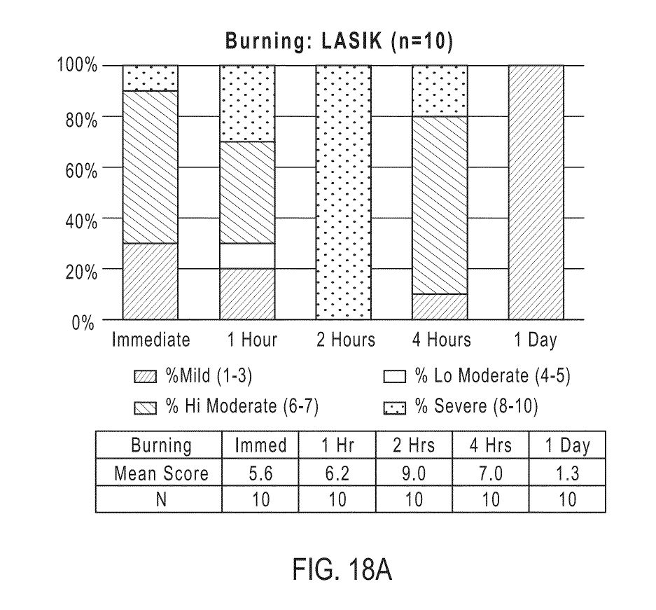

The visual acuity following LASIK surgery was measured for patients wearing coverings having different curvatures and at various times following surgery. The data is summarized in FIG. 21. The data indicates that coverings having a flatter central fit, such as from −1.5D to about −3.0D than the curvature of the cornea improve monocular UCVA, particularly at 2 hours following LASIK surgery. The region of the central fit is defined as the central 3 mm diameter center of the covering and the corresponding 3 mm diameter center of the cornea to which the covering is applied.

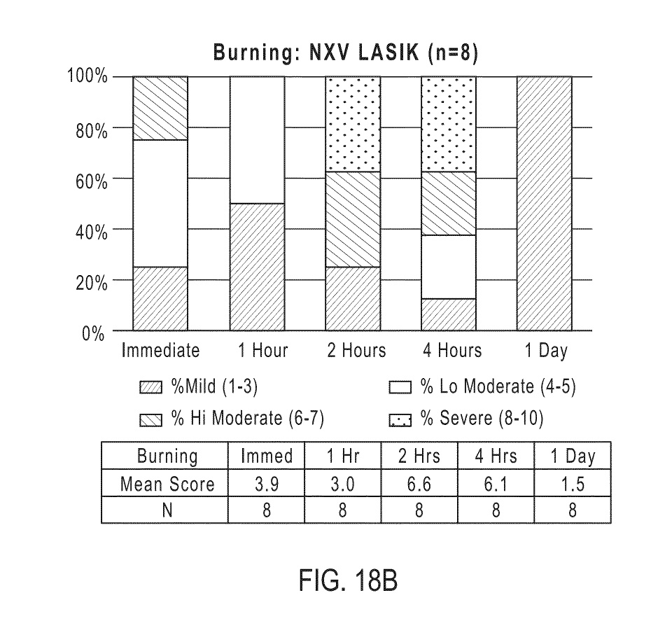

A flatter central fit also provides improved overall vision by avoiding a decrease in vision when the covering is removed from the cornea. In general, when a covering provided by the present disclosure is removed from the cornea, a patient experiences a temporary decrease in vision that lasts from 0 minutes to 180 minutes. This decrease in vision occurs whether the covering is removed within the first 2 hours, at 2 hours, or up to 24 hours after the covering has been applied to a patient's eye. As shown in FIG. 22, coverings that have a curvature that is from about −2.0D to about −3.0D the curvature of the cornea exhibit less of a reduction in the UCVA when removed from the patient's eye compared to coverings having a steeper profile.

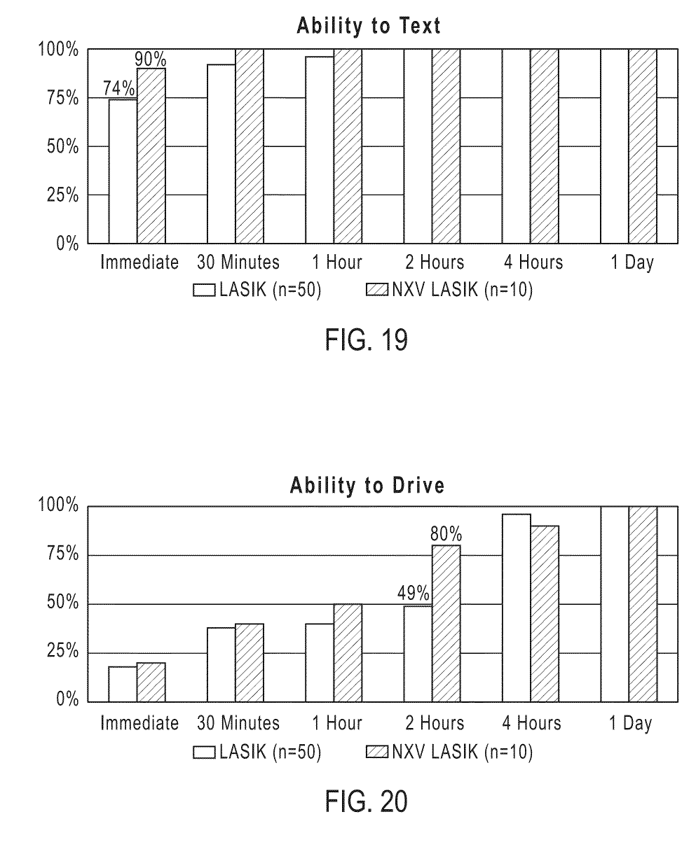

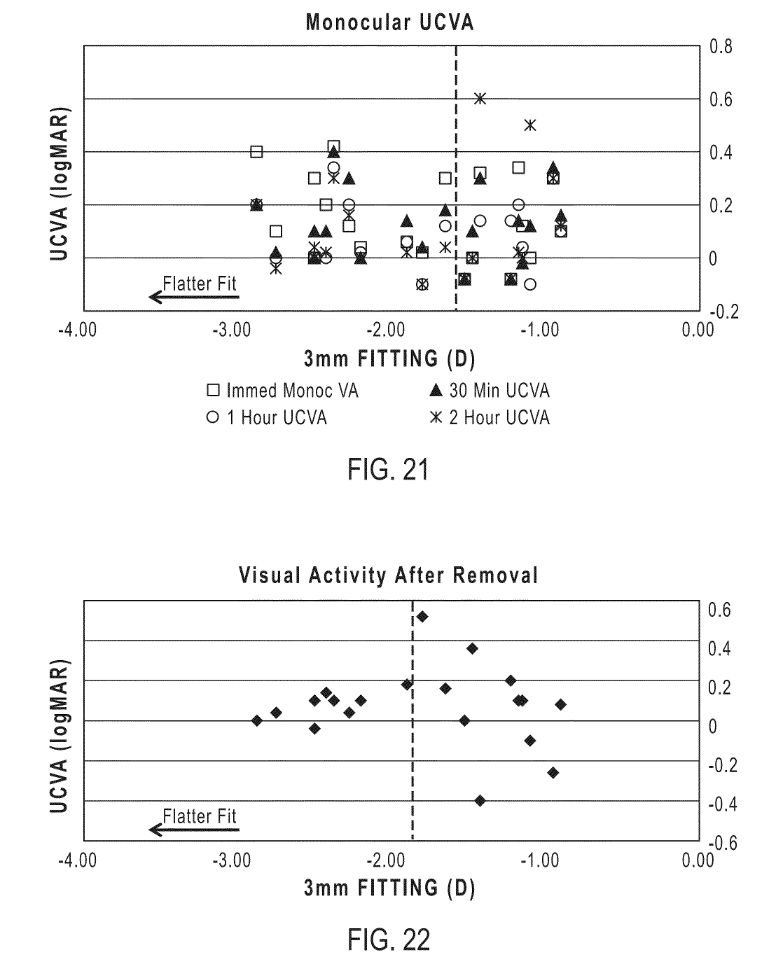

In addition to providing enhanced visual acuity, coverings having a flatter central fit can enhance the predictability of post-operative UCVA within a population of patients. As shown in FIG. 23, the UCVA of patients was determined at various times following PRK surgery. One group of patients wore coverings having a curvature equivalent to the corneal curvature (on-K), and a second group of patients wore coverings having a profile that was −1.0D to −2.5D the curvature (flat-K) of the cornea. In the group of patients wearing coverings having a steeper profile (on-K) the variation in the visual acuity among the patients as reflected by the error bars associated with the measured UCVA was greater than the variation in the visual acuity among the patients wearing coverings having a flatter curvature (flat-K). This difference was apparent at all times through 1 week following PRK surgery. Similar data is presented as a scatter plot in FIG. 24 for a study including 89 patients in which individual patients wore coverings having different profiles. A similar enhanced predictability of UCVA among patients following LASIK surgery can be expected.

It is believed that coverings having a steeper 5 mm mid-peripheral profile (curvature at 5 mm from the center of the covering, e.g., steeper than −1.0D, are more susceptible to trapping air bubbles during placement on the cornea, and that coverings having a flatter 5 mm fit, e.g., flatter than −2.5D, may slow epithelial healing. However, it has also been determined that coverings having a flatter 5 mm mid-peripheral fit, e.g., from −0.01D to −1.5D result in improved comfort at 2 days following ocular therapy. Issues concerning potential bubble entrapment may be addressed using the inverted placement technique for placing a covering on a patient's eye.

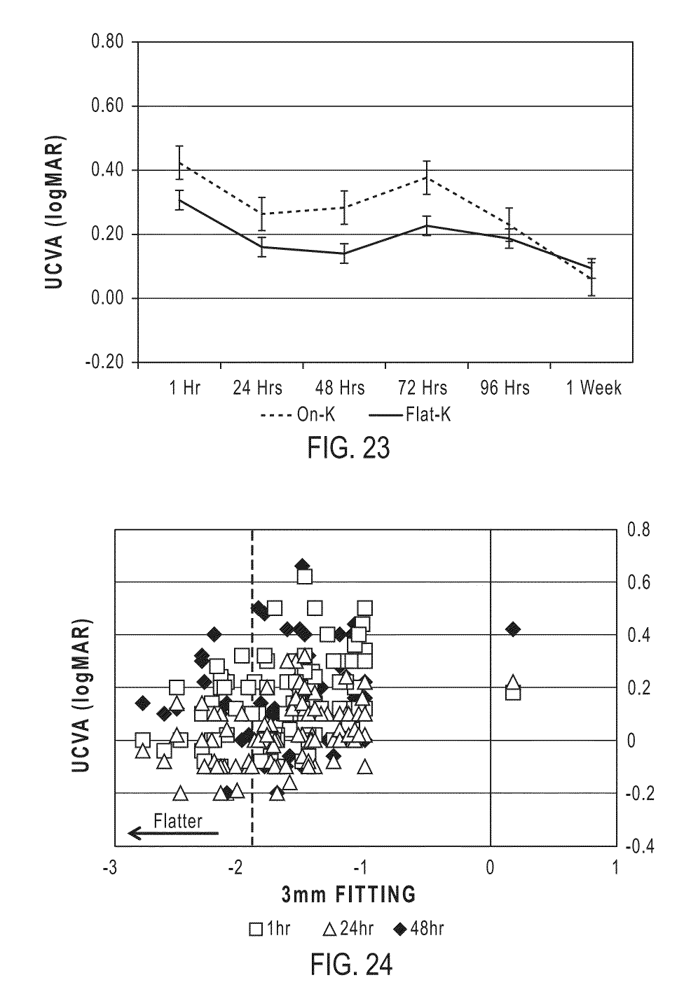

The ability of coverings having a flatter 5 mm (i.e., at an annular region of the covering about 5 mm from the centerline) fit to improve post-therapy comfort is presented in FIG. 25, which shows a general trend toward improved 48-hour post-PRK comfort with coverings having a flatter 5 mm mid-peripheral fit, such as for coverings that are 1.5D to 3.0D flatter than the cornea, e.g., −1.5D to −3.0D coverings. A similar correlation between mid-periphery fit and comfort is expected for coverings provided by the present disclosure used following ocular therapy.

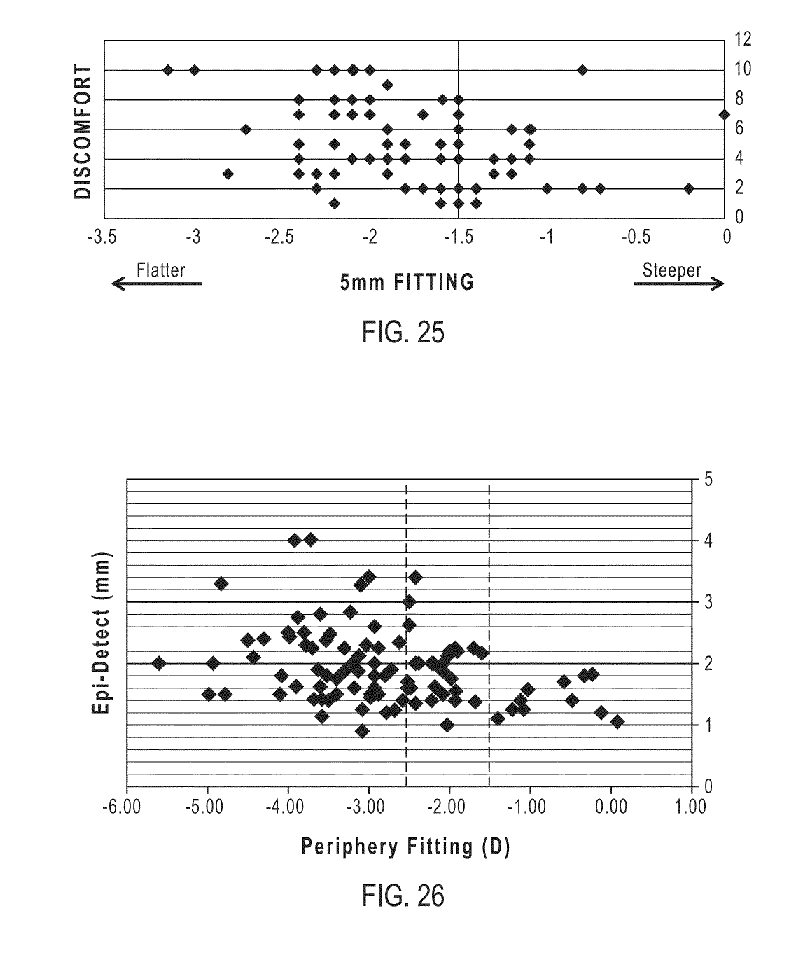

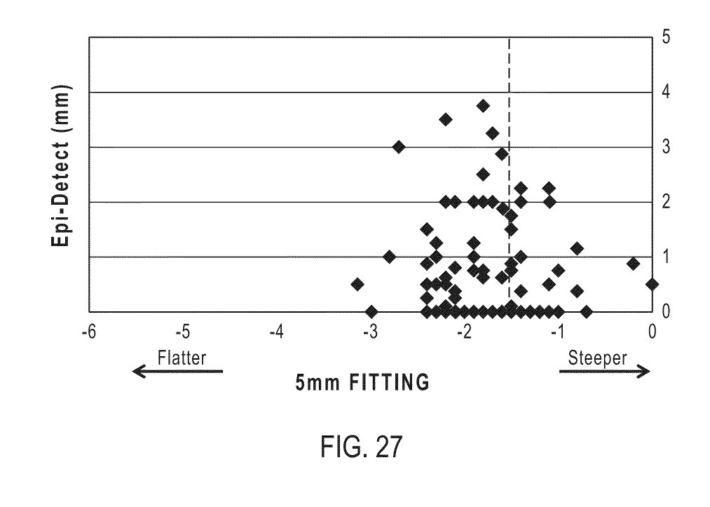

Factors associated with selecting an appropriate mid-periphery 5 mm fit include selecting a mid-peripheral curvature and/or shape to optimize epithelial healing and to minimize the entrapment of air bubbles during placement. The number of epithelial defects was measured after coverings having different mid-peripheral curvatures were applied to eyes following PRK surgery. As shown in FIG. 26, the number of post-PRK epithelial defects decreased for more steeply curved coverings. FIG. 27 shows the number of post-PRK epithelial defects for coverings having from about 0 to about −3.0 peripheral curvature, and indicates that a mid-peripheral curvature of less than −1.5D can provide improved performance by enhancing epithelial healing and by minimizing the entrapment of air bubbles. Although LASIK does not involve the extent of epithelial damage as in PRK; nevertheless, in LASIK the epithelium is transected to create a flap to enable access to the stroma. When the corneal flap is replaced over the ablated stroma, the transected epithelium must heal. In fact one of the complications of LASIK is epithelial ingrowth in which epithelial cells begin to grow underneath the corneal flap. It is estimated that about 30% of LASIK retreatment procedures have epithelial ingrowth issues. Therefore, appropriate selection of the mid-periphery 5 mm fit is expected to benefit epithelial recovery in LASIK patients, as was demonstrated for PRK patients.

When a covering is applied to an eye of a patient, the inner portion deforms or flexes to optically conform to the anterior surface of the cornea. This is shown, in part, in FIG. 4A and FIG. 4B. FIG. 4A shows a simplified cross-section of a covering 400 in which the inner surface 403 is characterized by a radius of curvature 401R. FIG. 4B shows a cross-section of an eye following LASIK surgery in which covering 400 is applied to the anterior surface of the cornea. When applied onto the anterior surface of the cornea, the inner surface 403 of the covering 400 optically conforms to the anterior surface of the cornea, which is characterized by a post-ablation profile. During application, the covering flexes such that the inner surface of the covering flattens, e.g., the radius of curvature of the inner portion increases and then takes the shape of the ablated corneal profile. In the embodiment illustrated in FIG. 4B following LASIK, there are no defects to the anterior surface of the cornea, as there would be in PRK surgery. In the absence of defects to the anterior surface of the cornea, a covering when applied to the eye, physically conforms to the anterior surface of the cornea, or more accurately, the anterior surface of the cornea physically conforms to the smooth optical surface of the covering.

The outer portion of a covering is generally configured to at least partially conform to the surface of the cornea and the peripheral portion or edge of the outer portion is configured to engage the epithelium, the sclera, or a combination thereof, so as to resist movement of the inner portion when the covering is placed on the eye. When applied to the anterior surface of the cornea the inner portion of the covering is configured to optically conform to the anterior surface of the cornea and in particular to the optical portion of the anterior surface of the cornea. The anterior surface of the cornea may comprise the epithelium, the stroma, or a combination of the epithelium and the stroma. When applied to the anterior surface of the cornea, the inner portion optically conforms to the anterior surface of the cornea, smoothing and sealing the corneal surface. The covering can smooth irregularities of the corneal tissue including the epithelium and/or sclera caused by the optical therapy. The covering seals the cornea from fluid transport, thereby reducing edema.

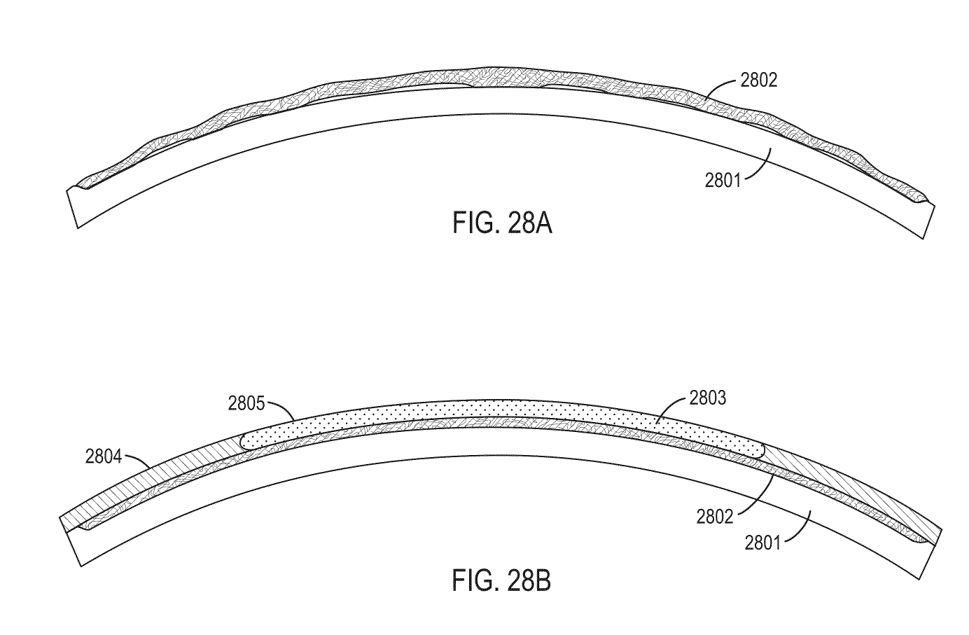

FIG. 28A shows a schematic cross-section of a post-LASIK cornea including stroma 2801 and edematous flap 2802. Edematous flap 2802 does not lie smoothly over ablated stroma 2801 causing surface irregularities. These surface irregularities can be measured using a corneal topographer as shown in FIG. 7A and FIG. 7B.

FIG. 28B shows a schematic of a post-LASIK cornea including ablated stroma 2801 and edematous flap 2802 with a covering 2803 having inner portion 2805 and outer portion 2804 applied to the anterior surface of the cornea. Application of the covering 2803 causes the edematous flap to conform to the inner surface of the covering and to conform to the profile of the ablated stroma such that the previous irregularities are smoothed.

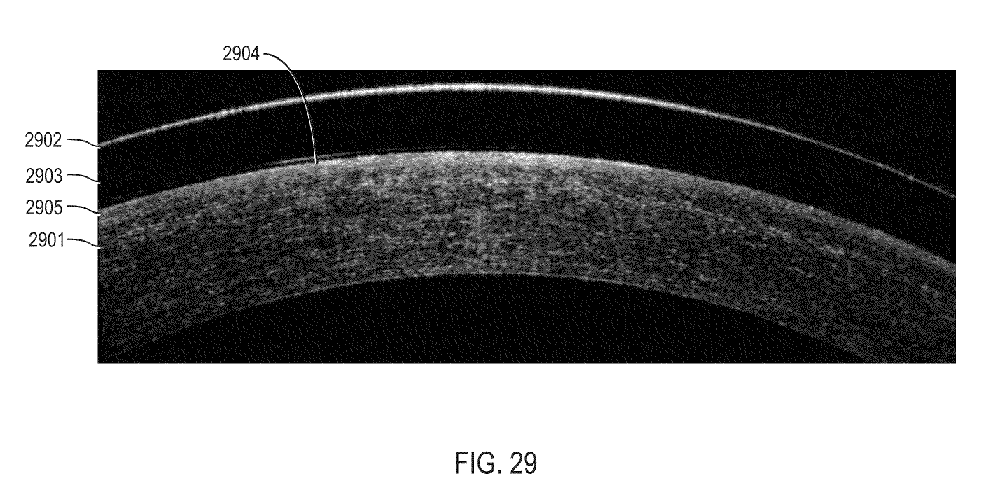

FIG. 29 shows a photograph of a cross-section of a cornea following LASIK with a covering applied to the anterior surface of the cornea. The inner surface of the covering conforms and smooths the anterior surface of the cornea. The covering provides the combined benefits of smoothing and stabilizing the optical surface, which improves visions, restores corneal physiology, which minimizes edema, promotes epithelial regrowth, and promotes healing of damaged ocular tissue. FIG. 29 shows the stroma 2901, the flap 2905, the covering 2903 having an outer surface 2902 and inner surface 2904.

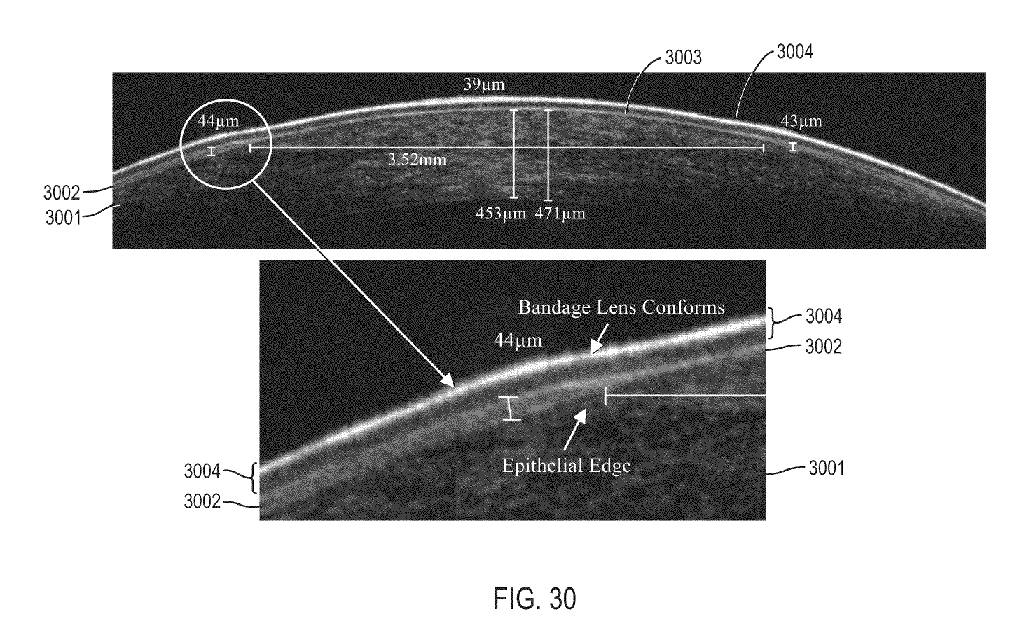

Bandage contact lenses are thin, soft contact lenses used to improve patient comfort and to promote epithelial healing of an injury to the eye following ocular surgeries such as PRK and LASIK. FIG. 30 shows a bandage lens applied to a post-PRK cornea. FIG. 30 shows a cross-section of a post-PRK cornea including ablated stroma 3001, healthy, undamaged epithelium 3002, ablated surface 3003, and bandage lens 3004 overlying the ocular tissue. Following PRK, the epithelium across the optical region of the cornea has been ablated. Healthy, 44 micron-thick epithelium 3002 is present toward periphery of the ablated region 3003. A bandage lens 3004 applied to the cornea physically conforms to and rests against the undamaged epithelium 3002 and the ablated stroma 3003. During recovery, the epithelium regrows from the periphery toward the center and during re-growth, the thin bandage lens continues to physically conform to the changing epithelial surface profile, causing the refractive properties of the eye to change during post-surgical healing. The inset shows a magnification of the interface between the undamaged epithelium and the ablated regions, clearly showing the migration of the re-growing epithelium into the ablated region. The physical conformance of the bandage lens to the topography of the epithelium is also clearly shown in the inset. In contrast, a covering provided by the present disclosure partially conforms, or more specifically optically conforms, to the epithelium.

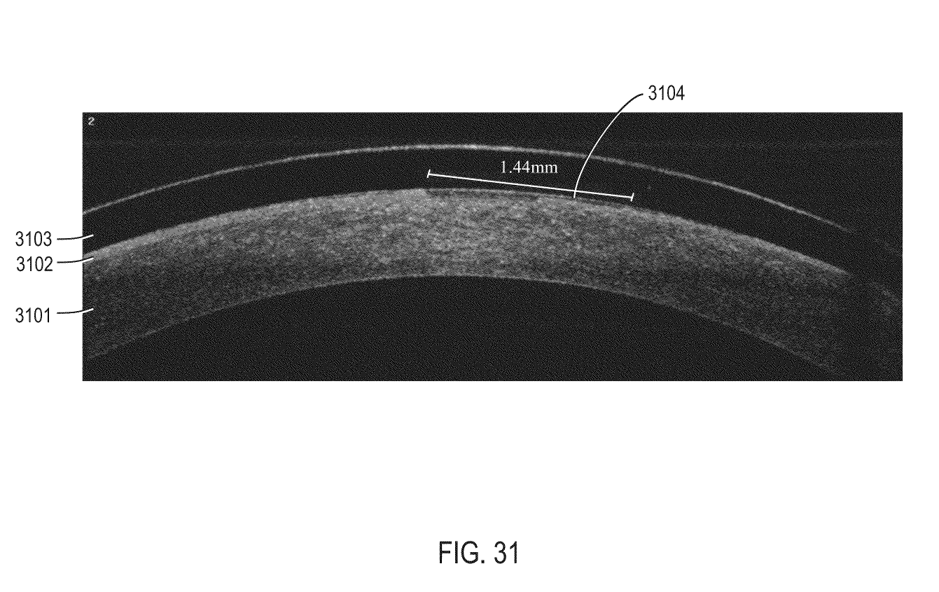

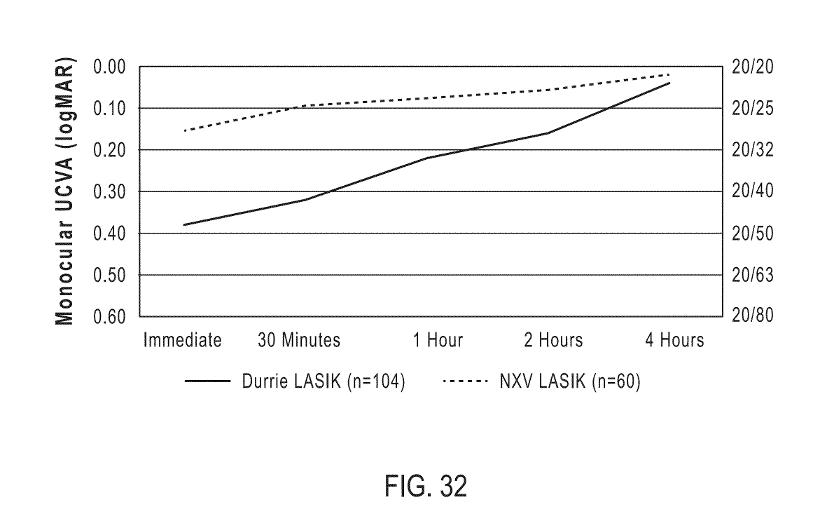

A covering conforms to the healthy, non-ablated epithelium and bridges and does not physically conform or rest against the surface of the stroma and/or damaged epithelium in the ablated region. Effectively, a covering bridges the gap due to the epithelial defect to form a lenticular region. In this sense, a covering optically conforms to the anterior surface of the cornea to provide a smooth, stable optical surface that does not change during corneal healing. The covering thus conforms to the overall optical shape of the treated cornea to provide a smooth optical surface. This is illustrated in FIG. 31, which shows an optical image of a cross-section of a cornea, including a stroma, healthy epithelium, and a 1.44 mm epithelial gap two-days following PRK. FIG. 31 shows a cornea including a stroma 3101, undamaged epithelium 3102, a 1.44 mm epithelial defect 3104, and an overlying covering 3103. The inner surface of the covering optically conforms to and rests on undamaged portions of the epithelium. In this way, a covering conforms to the overall post-therapy shape of the treated eye and thus provides stable and predictable vision during the recovery period.

In particular, the covering provides a lenticular region between the inner surface of the covering and the stroma and/or damaged epithelium, effectively spanning the anterior region of the cornea spanning healthy epithelium that provides a physiological environment that promotes epithelial growth and provides a physical scaffold to guide and smooth epithelial regrowth. During epithelial regrowth, the epithelium regrows from the perimeter of the lenticular region toward the center. Because the inner surface of a covering does not physically conform to the epithelial defect, the profile of the inner portion of the covering does not change during recovery. In this way, the covering provides a stable optic during recovery.

From a functional perspective, when applied to the anterior surface of the eye, coverings provided by the present disclosure seal the anterior surface of the eye and smooth the optical region of the eye. Coverings at least partially or fully conform to the anterior surface of the eye and engage the peripheral portion of the eye such as the epithelium and/or sclera to resist movement of the optical or inner portion of the covering. The seal against the eye facilitates restoration of normal corneal deturgescence following corneal manipulation and resists edemal pressure. By controlling and/or minimizing the transient post-manipulation edema, visual recovery is accelerated.

The seal applied by a covering can also serve to join ocular membranes and/or ocular inserts following ocular manipulation. For example, incisions through optical tissue and ocular inserts such as inlays produce interfaces that seal during recovery. Application of a covering following ocular manipulation can serve to stabilize these interfaces and thereby accelerate healing and avoid complications associated with incomplete wound healing such as leakage.

Topographical corneal irregularities that at least in part are associated with edema and/or ocular manipulation are also smoothed by use of a covering provided by the present disclosure. Soothing of the transient corneal irregularities in the optical region can lead to faster visual recovery.

In certain embodiments, coverings provided by the present disclosure may be used to promote epithelial healing. The center portion of a covering is configured to at least partially contact the non-optical anterior surface of the cornea and toward the peripheral edge of the outer portion, engaged the epithelium, sclera, or combination thereof. In certain embodiments, the outer portion is configured to smooth the non-optical anterior surface of the cornea. Engagement of the peripheral portion resists movement of the inner portion and seals the peripheral portion of the covering.

Thus, in certain embodiments, which applied to the eye, a covering provided by the present disclosure optically confirms to the optical portion of the cornea, physically conforms to the non-optical portion of the cornea, engages the epithelium, sclera or combination thereof to resist movement of the inner portion, and seals the anterior surface of the cornea.

In certain embodiments, a primary function of a covering is to speed healing of ocular tissue and secondarily to improve visual acuity. In such embodiments, where ocular healing is the primary objection, a covering seals the eye and stabilizes ocular tissue by reducing motion and holding opposing ocular tissue in place. Furthermore, for certain healing applications, a covering my not include a rigid center portion. For example, a covering for ocular wound healing applications may be made from a single material characterized by a certain modulus. Such coverings seal the eye and may conform to the anterior surface of the cornea such that the covering does not bridge epithelial defects. The physical properties of the material used to form the covering and the shape of the peripheral edge can provide a seal that speeds wound healing. A covering may further speed healing by molding and/or smoothing irregularities of the epithelium, cornea, and or other ocular tissue to speed healing. For example, in a covering formed from a single material, the covering may have the same or similar dimensions to a covering having a central rigid portion and the modulus of the material may be from 0.1 MPa to about 40 MPa; the material may be selected from a silicone polymer, a silicone hydrogel, and a combination thereof; the material may have a water permeability less than about 0.2 cm²/sec; and the material may have an oxygen permeability of at least about 300 Dk.

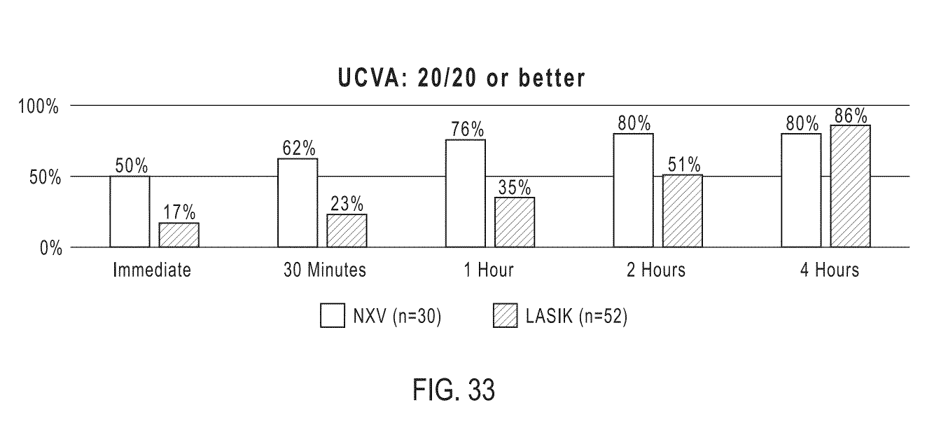

FIG. 33 is a graph comparing the monocular UCVA (log MAR) in a population of patients wearing a covering (NXV LASIK n=60) with a population of untreated LASIK patients (Durrie LASIK n=104). The data shows that the covering provide significantly improved UCVA when applied immediately following LASIK surgery and during the first about four hours following LASIK surgery.

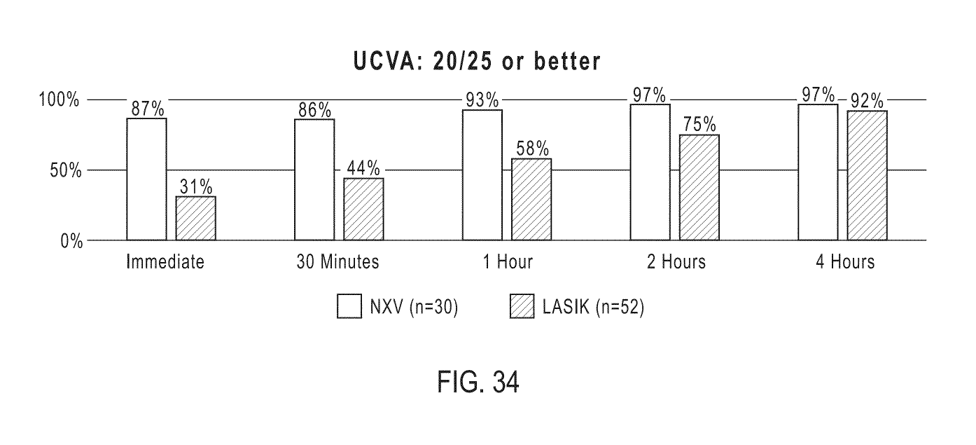

FIG. 34 shows histograms comparing the percent of patients having a UCVA of 20/20 or better at various times following LASIK when wearing a covering (NXV n=30) and untreated (LASIK n=52).

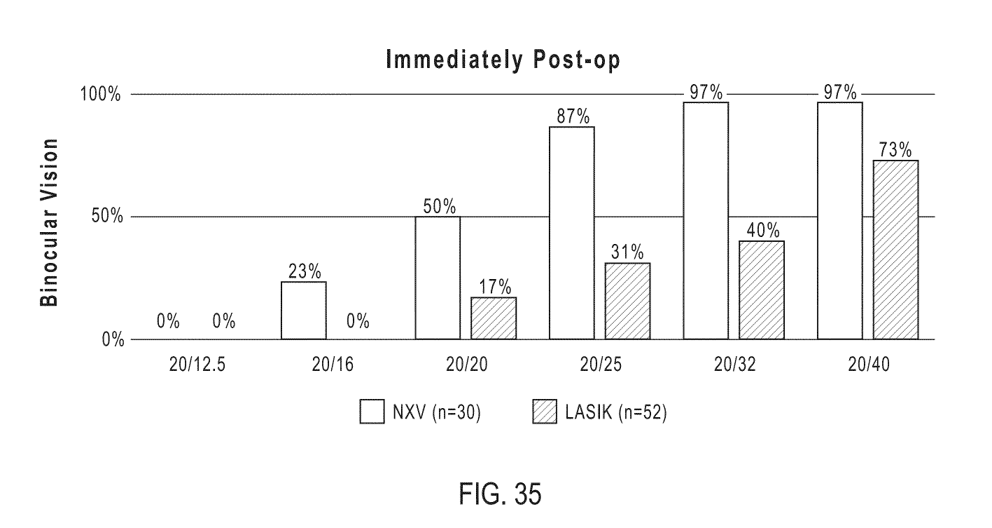

FIG. 35 shows histograms comparing the percent of patients having a UCVA of 20/25 or better at various times following LASIK when wearing a covering (NXV n=30) and untreated (LASIK n=52).

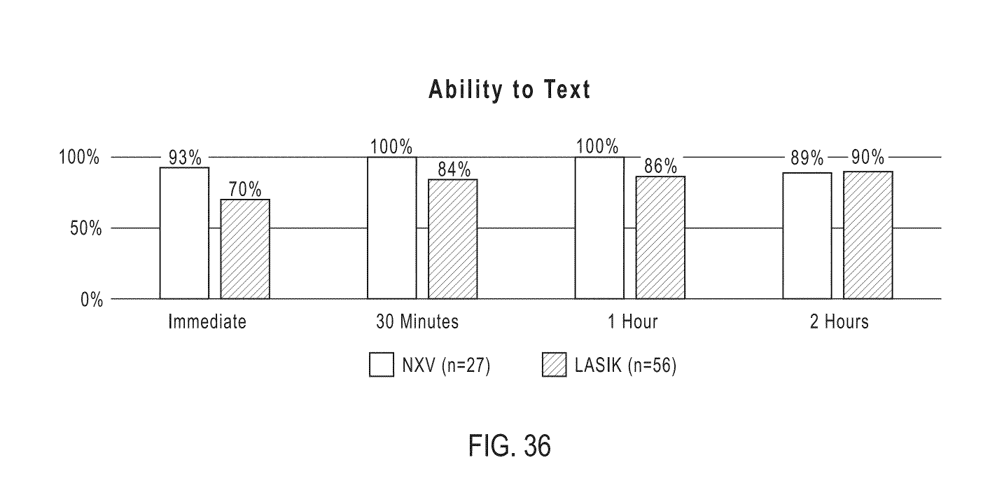

FIG. 36 shows histograms comparing the percent of patients having a certain binocular visual acuity immediately following LASIK when wearing a covering (NXV n=30) and untreated (LASIK n=52).

The results presented in FIGS. 34-36 indicate that wearing a covering provided by the present disclosure immediately following LASIK provides immediate visual recovery.

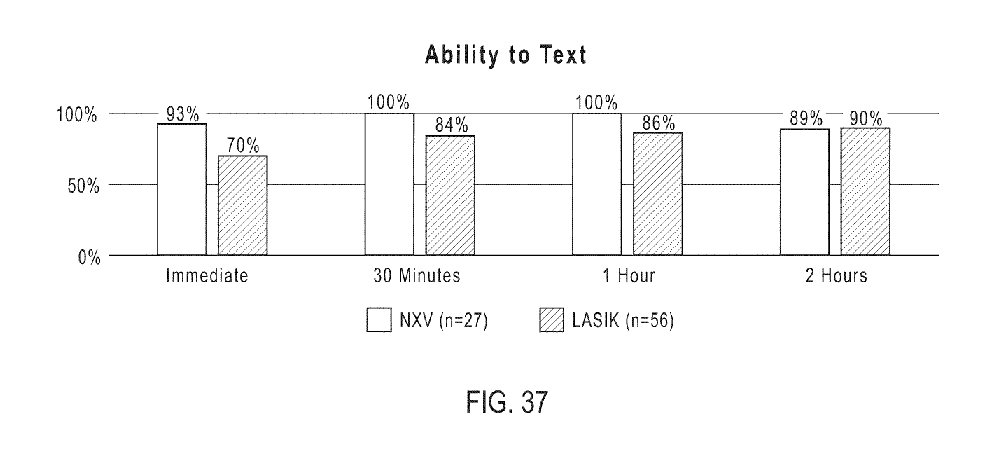

FIG. 37 shows histograms comparing the percent of patients able to text using a mobile telecommunications device at various times following LASIK when wearing a covering (NXV n=27) and untreated (LASIK n=56).

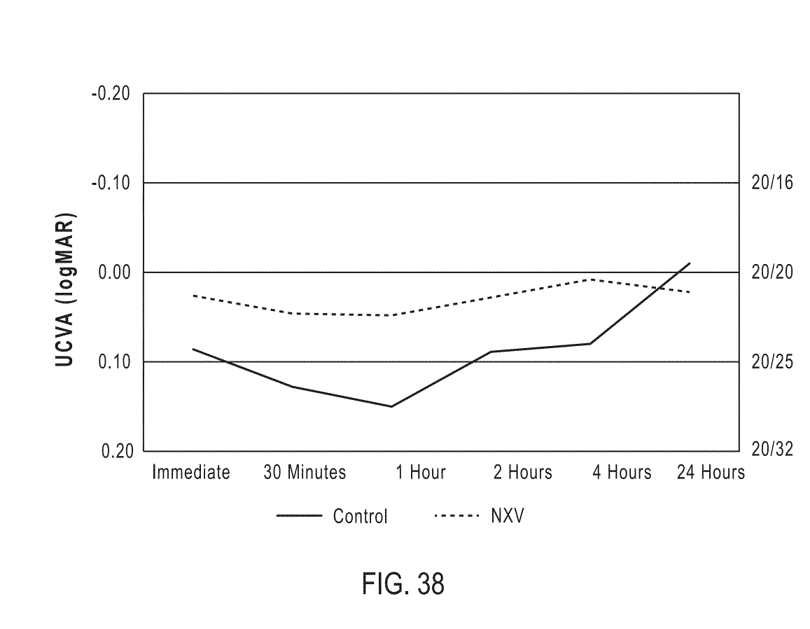

FIG. 38 shows histograms comparing the percent of patients able to drive at various times following LASIK when wearing a covering (NXV n=43) and untreated (LASIK n=57).

The results presented in FIG. 37 and FIG. 38 indicate that wearing a covering provided by the present disclosure immediately following LASIK improves functional recovery.

A contra-lateral study was undertaken to compare visual and functional recovery following LASIK in an eye wearing a covering provided by the present disclosure and in an eye wearing a bandage contact lens.

LASIK surgery was performed on both eyes in a population of patients and immediately following surgery a covering was applied to one eye and a bandage contact lens (Optix Night&Day) was applied to the other eye. Post-LASIK UCVA, discomfort, and patient preference were determined.

FIG. 38 is a graph of the UCVA during the first 24 hours following LASIK. In the eyes with the covering, near 20/20 vision was realized upon application and was maintained during the 24 hour measurement period. In contrast, in the eyes with the bandage contact lens 20/20 visual recovery was not restored until more than 4 hours post-surgery.

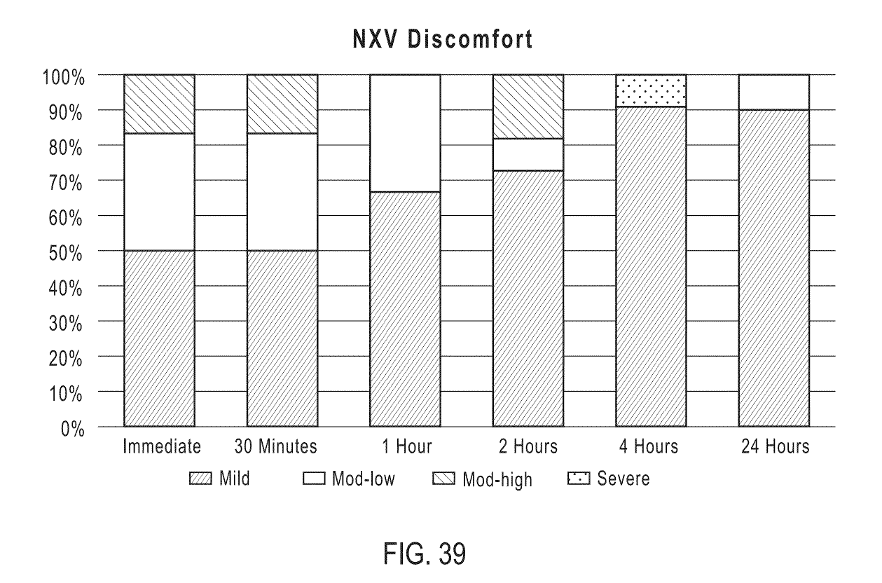

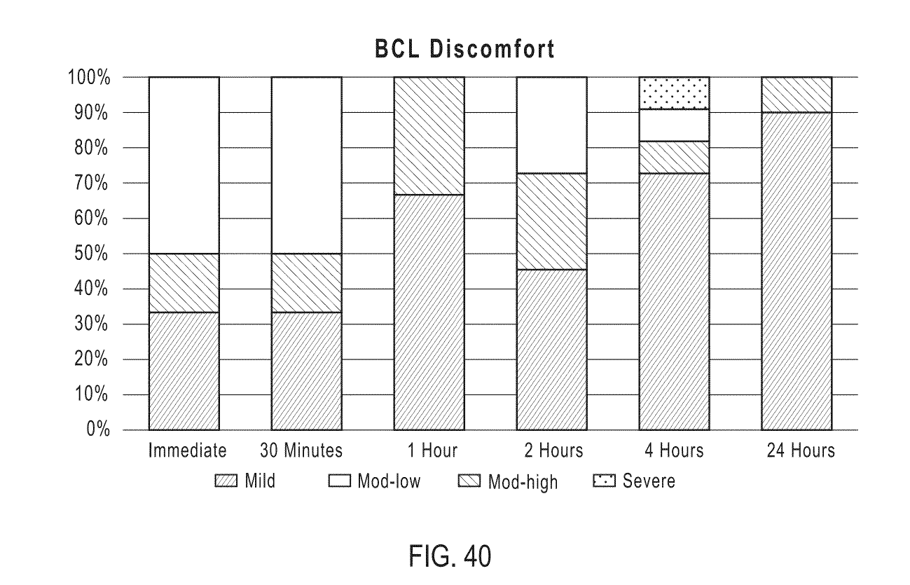

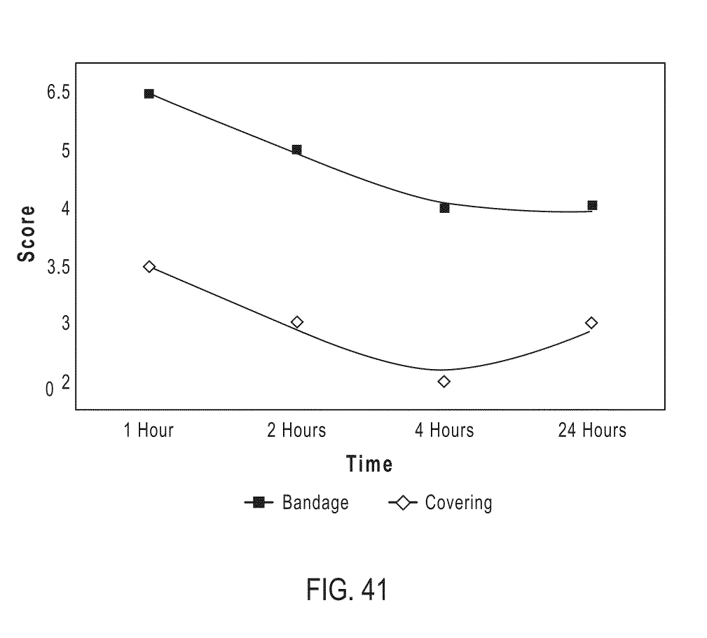

Assessment of overall discomfort including pain, light sensitivity, foreign body sensation, and heavy lid sensation was also determined. As can be appreciated by comparing the histograms of the results presented in FIG. 39 for eyes with the covering and FIG. 40 for untreated eyes, patients perceived less overall discomfort in eyes wearing a covering during at least the first 4 hours following LASIK compared to eyes wearing a bandage lens. The data was also evaluated using a weighted analysis method was used to emphasize the perceived differences in overall discomfort. The results are presented in FIG. 41, which shows that patients perceive a markedly improved overall comfort, as reflected in the lower overall discomfort scores, in eyes wearing a covering compared to eyes wearing a bandage lens, and that the improved comfort persisted at least during the first 24 hours following LASIK.

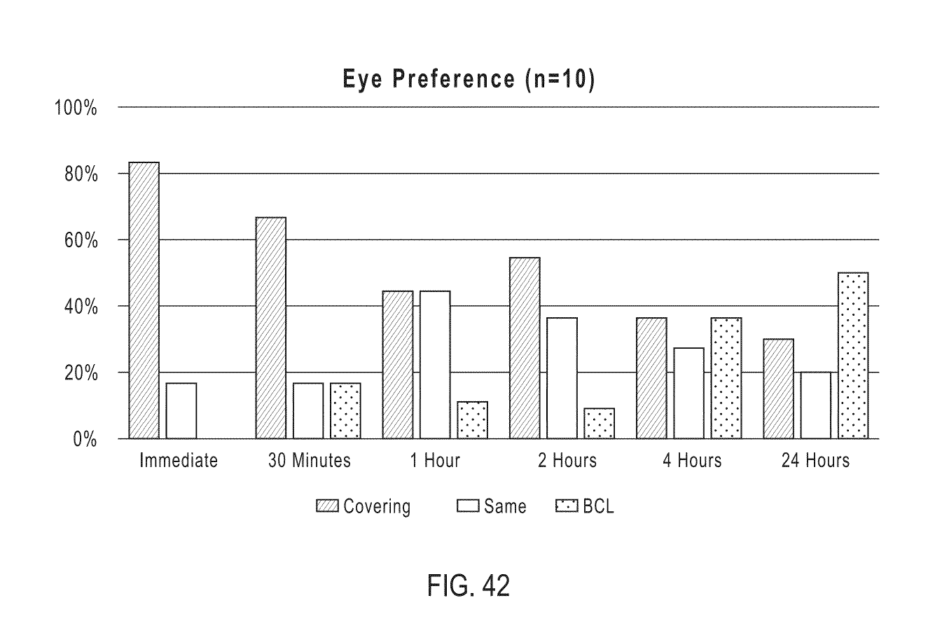

Not surprisingly given these results in which post-LASIK vision and comfort were improved during at least the first several hours following surgery, as confirmed by the results presented in FIG. 42, patients expressed a preference for the covering compared to a bandage lens for post-LASIK treatment immediately following LASIK and at least during the first couple of hours.

In post-PRK treatment, a covering does not physically conform to the entire anterior surface of the cornea, but rather bridges epithelial defects to optically conform to the cornea and provide an optically smooth surface.

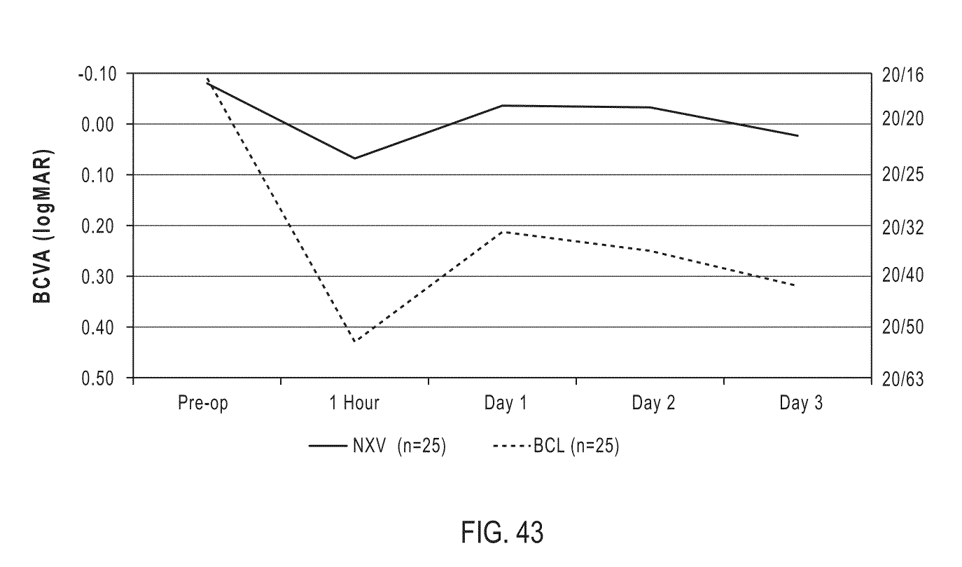

The ability of coverings provided by the present disclosure to improve the optical quality of the ocular surface is reflected in the improved BCVA following PRK. In a contralateral study, PRK patients wore either a covering (NXV n=25) or a bandage contact lens (PRK BCL n=25) on one of the eyes. The results are shown in FIG. 43. The BCVA (log MAR) for eyes wearing a covering was 20/20 within 1 hour following PRK surgery and throughout the study period. In contrast the BCVA for eyes wearing the bandage contact lens was significantly reduced at 20/50 with reduced BCVA on the order of 20/40 persisting at least during the 3 days of the study period. The comparison was significantly significant at all times tested (p<0.05 T-test). BCVA is at least in part determined by corneal surface topography, and the improved BCVA while wearing a covering can be attributed to the ability of the covering to provide smooth ocular surfaces. In contrast, because the thin bandage contact lens, completely conforms to corneal surface irregularities, which are apparent during the surgical recovery period, additional optical correction is not possible.

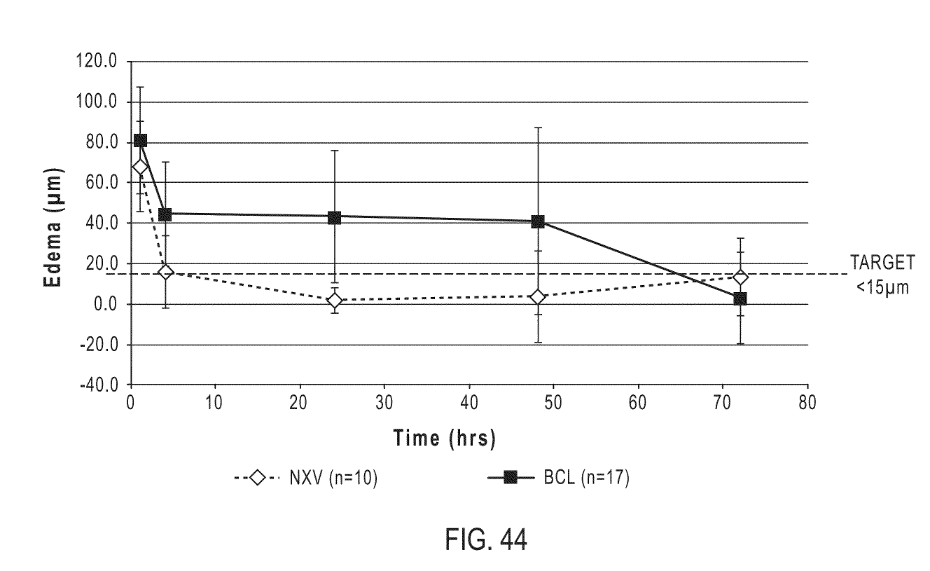

The ability of the covering to resolve post-operative stromal edema is shown in FIG. 44. FIG. 44 shows the results of a contralateral study in which the post-PRK edema was measured in eyes wearing a covering and in eyes wearing a bandage contact lens. As shown in FIG. 44, when a covering is applied to an eye immediately following PRK, edema is reduced to 15 μm during the first few hours and remains below about 15 μm during the first 72 hours following surgery. In contrast, although there is some reduction in post-PRK edema with the bandage contact lens, edema remains high at least during the first 48 hours following surgery. The differences at all times test were statistically significant (p<0.0001). Improved visual acuity when wearing a covering can therefore be attributed in part to the ability of the covering to resolve and immediately dissipate post-surgical edema and thereby provide a smooth optical surface.

In general, coverings provided by the present disclosure provide the following benefits for post-PRK and post-LASIK treatment: (1) minimize treatment fluctuation with high rigidity optic; (2) minimize optical aberrations/irregular corneal surface with high rigidity optic; and 93) minimize light scatter by accelerated edema resolution.

Coverings provided by the present disclosure are able to mitigate and resolve edema by providing a barrier to fluid flow to and from the anterior surface of the cornea. Coverings do not have any appreciable water content and are configured to form a seal with the cornea and/or sclera at the periphery. In contrast, bandage contact lenses do not form a seal.

The environment and structure of the lenticular region bridging the epithelial defect also stimulates epithelial regrowth. Epithelial regrowth was monitored following PRK in eyes wearing a covering to provide a lenticular region, and in eyes wearing a bandage contact lens that conformed to the topography of the healthy epithelium as well as to the ablated stroma such that there is not a lenticular region. As shown in Table 1, the epithelium was completely healed in more eyes with the covering during the first few days following surgery compared to eyes with a completely conforming bandage lens.

TABLE 1

| Cumulative % | Day 1 | Day 2 | Day 3 | Day 4 |
|---|---|---|---|---|
| Covering | 0% | 0% | 52% | 92% |
| (n = 25 eyes) | | | | |
| Bandage contact lens | 0% | 0% | 32% | 72% |
| (n = 25 eyes) | | | | |

The effects observed for ocular post-surgical recovery such as following PRK and LASIK surgery can be expected to pertain to ocular wound healing in general. Ocular wounds can be associated with, for example, edema, corneal irregularities, and defects involving damaged epithelium. These aspects, which are addressed by the coverings in post-LASIK and post-PRK treatment can be addressed when applied to healing of ocular wounds of any origin, including surgical and non-surgical wounds. Coverings provided by the present disclosure exhibit high oxygen permeability, which promotes ocular health, provide a stationary seal to mitigate fluid transport and thereby resolve edema and stabilize ocular tissue during the healing process, and provide a rigid scaffold that optically conforms to the cornea but not to epithelial defects and thereby provides a lenticular region having an environment and structure that is effective in stimulating epithelial regrowth.

Coverings provided by the present disclosure can also reduce the risk of diffuse lamellar keratitis (DLK). DLK is a nonspecific sterile inflammatory response that can result from refractive surgeries such as LASIK. Symptoms range from mild photophobia and/or mild decreased vision to severe decreased vision. Intraoperative epithelial defects are considered to be one of the causes of DLK. The ability of a covering to facilitate the ability of the LASIK flap to adhere to the ablated cornea and to restore the epithelium following surgery can reduce DLK caused by epithelial defects and others.

In summary, coverings provided by the present disclosure at least provide improved vision following optical therapy, increase post-therapy comfort, and increase the rate of functional visual recovery. These attributes and others have been demonstrated for LASIK and PRK. Similar improvement in vision, healing, and patient comfort are expected to be realized in other ocular therapies involving manipulation of the cornea. These improvements are particularly apparent during the first two hours following optical therapy.

A covering may be removed at any appropriate time after being applied to a patient's eye, which may at least in part be determined by the judgment of the physician and/or the convenience of the patient. For example, a covering may be removed 2 hours, 4 hours, 8 hours, 12 hours, 24 hours, or any other time following application. In certain embodiments, a covering is removed about 4 hours following ocular therapy. It has been observed that when a covering is removed from an eye within a few hours following application and during the recovery period, such as about 2 hours following therapy and application, patient comfort may be compromised. For example, when a covering is removed about 2 hours following ocular therapy and application, a patient may experience photophobia and a burning sensation similar to that of a patient without a covering. Nevertheless, this discomfort is temporary, and is less severe than is experienced by a patient without the covering applied to the eye. Therefore, in certain methods, a covering may be applied to an eye immediately following ocular therapy and removed within the first two hours and still achieve benefits of enhanced vision and improved comfort.

A covering may be removed from the eye by the physician, by other medical personnel, or by the patient. In general, it is desirable that a covering be removed by grasping the outer periphery of the covering and pulling the covering from the eye.

Known corneal correction treatment methods have generally been successful in correcting standard vision errors, such as myopia, hyperopia, astigmatism and the like. However, as with all successes, still further improvements would be desirable. Toward that end, wavefront measurement systems are now available to measure the refractive characteristics of a particular patient's eye. By customizing an ablation pattern based on wavefront measurements, it may be possible to correct minor regular and/or irregular refractive errors so as to reliably and repeatably provide visual acuities of 20/20 or better. Unfortunately, these measurement systems are not immune from measurement error. Similarly, the calculation of the ablation profile, the transfer of information from the measurement system to the ablation system, and the operation of the ablation system all provide opportunities for the introduction of errors, so that the actual long term visual acuities provided by real-world wavefront-based correction systems may not be as good as might be theoretically possible.

Various embodiments of the invention provide methods and systems for verifying procedures used to correct aberrations in the eye resulting in vision defects such as myopia, etc. Particular embodiments are useful for pre-operatively verifying the effectiveness of laser eye surgical procedures such as photorefractive keratectomy (PRK), phototherapeutic keratectomy (PTK), laser in situ keratomileusis (LASIK), and the like, and in particular for presbyopic correction.

In certain embodiments, coverings provided by the present disclosure may be used as trial lenses or verification lenses for verifying prospective refractive correction to an eye of a patient. Because of uncertainties associated with refractive surgeries and due to the fact that the outcome can associated with a tradeoff between conditions of visual acuity and thereby involve patient preference, it can be useful for a patient to experience the expected results of refractive surgery prospectively. Trial or verification lenses can provide a patient to experience the expected results prior to a surgery. In certain embodiments, a method comprises measuring irregular aberrations of the eye. A determination is made for a proposed refractive correction for treatment of the eye. The determination can be based on the measured aberrations or other optical evaluation of the eye. A central portion of a verification lens is configured to correspond with the proposed correction. A peripheral portion of the verification lens is positioned upon the sclera of the eye so that the central portion is optically aligned with the aberrations. This can be accomplished by registering the verification lens with the eye. Then a determination is made whether the corrected vision of the eye with the verification lens is acceptable. This determination is used to verify the proposed correction. The determination can include an evaluation of one or more of visual acuity, accommodation and contrast sensitivity as well the reading of an eye chart. The determination can be made after the verification lens has been worn for a period of hours, a day or even multiple days. Also, several determinations can be made over a desired period and the results compared (e.g., by quantitative or qualitative means). Various embodiments of the method can be used to evaluate a number of eye treatments including laser refractive treatments and the like. Also, in many embodiments, the irregular aberrations or other optical errors of a patient's eye can be measured with a wavefront sensor which, in specific embodiments, can be configured to measure refractive error. Measurements from the wavefront sensor can be used to produce a wavefront shape which can be used to configure the verification lens to correspond to the proposed correction. For example, in one embodiment, the wavefront shape is used to generate an ablation pattern (described below) for fabrication of the verification contact lens or a corrective contact lens worn by the patient on a long term basis.

In various embodiments, a treatment portion of the trial lens can have an aspheric shape configured to correspond to a proposed correction to treat various conditions of the eye such as refractive errors, higher order aberrations and presbyopia. Typically, the treatment portion comprises a central portion of the lens, but can comprise a non-central portion or even the entire lens.

In various embodiments, the peripheral portion of the lens can be configured to stabilize or otherwise reduce movement of the verification lens. For example, in one embodiment, the peripheral portion is used to stabilize the verification lens during determination of the corrected vision. This can be accomplished by configuring the peripheral portion to have a surface contour corresponding to a surface contour of the sclera so that the peripheral contour stabilizes the verification lens on the eye. The peripheral portion can also be used to reduce movement of the verification lens such as that which may result from blinking, eye movement (e.g., nystagmus) or head movement or a combination thereof. Also, the peripheral portion can be used to facilitate registration by supporting a substantial portion of the verification lens on the eye.

In various embodiments, the verification pattern can be an ablation pattern. The ablation pattern can be generated based on a proposed refractive correction treatment of the eye. The ablation pattern for the verification lens can be calculated from the measured irregular aberration of the eye, and from characteristics of the lens material, such as a refractive index of the lens material, a rate of ablation of the lens material, and/or ablation properties of the lens material (e.g., the propensity of the lens material to differ in ablation depth across a uniform ablation energy beam, such as any “central island” properties of the lens material). A corneal tissue of the eye may also be ablated according to an ablation pattern, and the ablation pattern may similarly be calculated based on the measured optical error of the eye and on the corneal tissue characteristics, such as a refractive index of the corneal tissue, a rate of ablation of the corneal tissue, and/or a shape of ablation of the corneal tissue. In many embodiments, the wavefront shape can be used to generate the ablation pattern to produce a corrective scleral lens which can be worn by the patient on a long term basis similar to conventional correct contacts lenses known in the art. (e.g., daily wear, extended wear, etc.)

After an alignment/fitting determination, visual performance using the verification lens can be assessed. Visual performance assessment can be done immediately after the fitting determination, that same day after the patient has worn the lens for a number of hours or even after the patient has worn the lens for a number of days (e.g., two or more) though not necessarily continuously. The types of visual determination which can be made include without limitation, measurement of visual acuity (e.g., using a standard eye chart), depth of field, accommodation, contrast sensitivity and combinations thereof. One or more of these tests may be done under varying lighting conditions. The patient could also complete a subjective visual performance questionnaire. Information from one or more of these tests could be stored on a database and be used for evaluation of subsequent visual corrective plans for the particular patient, or a patient population or even a sub-population (e.g., pediatric patients or myopic patients). When using a scleral verification lens, prior to the visual assessment, the patient may register the verification lens with their eye by positioning the peripheral portion of the lens on the sclera so that the central portion of the lens is optically aligned with aberrations of the eye.

Visual performance of the verification lens may be assessed by having the patient scan an eye chart to determine visual acuity. If the measured visual acuity is equal to or better than some predetermined threshold value, often 20/20 or better and optionally 20/15 or better, the eye is ablated with the planned ablation pattern. If not, a second measurement may be taken and the process repeated, and if acuity still remains unacceptable, the ablation may not be performed.

After the corrective lens is fabricated, the wavefront measurement can also be repeated with the corrective lens in place on the eye, to verify the correction of the lens. The information from wavefront measurements with the lens in the eye, can also be used to titrate or fine tune the corrective profile of the corrective lens using lens fabrication methods described herein (e.g., lens ablation methods). A trial lens can be a soft or hard contact lens and can thus be fabricated using soft or hard contact lens materials and processing methods known in the art including gas permeable materials and technology. Also, a corrective lens can be configured to have an aspheric shape to correct for standard errors, such as refractive errors, as well as irregular errors such as higher order aberrations and presbyopia.

In certain embodiments, methods of treating an eye of a patient following an ocular therapy comprise, the eye comprising ocular tissue including a cornea having an anterior surface and an optical region used for imaging with the eye, an epithelium, and a sclera, the ocular therapy involving manipulation of the ocular tissue and associated with a lesion external to the optical region, comprise: providing a covering comprising: an inner portion characterized by an inner rigidity, and comprising an upper surface and a lower surface; and an outer portion characterized by an outer rigidity, and comprising an upper surface and a lower surface; wherein the inner rigidity is greater than the outer rigidity; following the ocular therapy, applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In certain embodiments, methods of treating an eye of a patient following an ocular therapy comprise, the eye comprising ocular tissue including a cornea having an anterior surface and an optical region used for imaging with the eye, an epithelium, and a sclera, the ocular therapy involving incising the ocular tissue and implanting a device within the optical region, comprise: providing a covering comprising: an inner portion characterized by an inner rigidity, and comprising an upper surface and a lower surface; and an outer portion characterized by an outer rigidity, and comprising an upper surface and a lower surface; wherein the inner rigidity is greater than the outer rigidity; following the ocular therapy, applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In certain methods, the inner portion optically conforms to the optical portion of the cornea.

In certain methods, the ocular therapy is selected from cataract surgery, corneal inlay surgery, corneal transplant surgery, and treatment of an ocular trauma wound.

In certain methods, the ocular therapy comprises incising the cornea and/or perforating the cornea at a site external to the optical region, and wherein the covering extends over the site.

In certain methods, the ocular therapy comprises cataract surgery.

In certain methods, following treatment with the method, leakage from the cornea is reduced compared to leakage from the cornea without treatment.

In certain methods, the inner portion provides a smooth optical surface and the covering reduces edema.

In certain methods, the covering, when applied to the anterior surface of the cornea, seals the anterior surface of the cornea.

In certain methods, the ocular therapy comprises corneal inlay surgery.

In certain methods, the method further comprises refractive surgery.

In certain methods, refractive surgery comprises LASIK.

In certain methods, the lower surface of the inner portion is characterized by at least one inner radius of curvature; and the outer portion comprises a peripheral portion having a lower peripheral surface characterized by at least one peripheral radius of curvature; and the methods further comprise engaging the adjacent epithelium with the lower peripheral surface of the covering so that the eye views through the inner portion.

In certain methods, the at least one inner radius of curvature comprises a radius of curvature corresponding to or less than a radius of curvature associated with the optical power of the stroma following ocular therapy; and the methods further comprise deforming the inner portion during the applying of the covering so that the upper surface of the inner portion optically conforms to the anterior surface of the cornea such that patient vision through the covering benefits from the therapy such that, within 4 hours of the therapy, patient discomfort associated with the therapy is mitigated and/or patient vision associated with the therapy is enhanced.

In certain methods, the outer portion comprises a peripheral portion and one or more intermediate portions, each of the one or more intermediate portions independently characterized by an intermediate radius of curvature; and the methods further comprise inhibiting movement of the inner portion relative to the eye by deforming at least the peripheral portion during the applying of the covering so as to promote motion-inhibiting engagement of the peripheral portion against the epithelium, the sclera, or a combination thereof, of the eye.

In certain methods, the inner portion is characterized by a substantially uniform thickness.

In certain methods, the inner portion is characterized by a shape that does not substantially correct vision.

In certain methods, the inner rigidity is from 1.2E⁻⁶ Pa-m³ to 3.1E⁻³ Pa-m³ and the outer rigidity is from 5.4E⁻⁹ Pa-m³ to 1.5E⁻⁴ Pa-m³.

In certain methods, the inner portion is characterized by an index of refraction that corresponds substantially to the index of refraction of the cornea.

In certain methods, the inner portion and the outer portion comprise a material selected from silicone, a silicone hydrogel, or a combination thereof.

In certain methods, following treatment with the method, at least about 50% of a population of patients see 20/20 or better within about 5 minutes following the ocular therapy.

In certain methods, in a population of patients, following use of the method, treatment is characterized by at least one of: stromal edema is reduced by at least about 25 μm within about 30 minutes following the ocular therapy compared to stromal edema without treatment; overall discomfort is reduced at least within the first 2 hours following the ocular therapy compared to overall discomfort without treatment; outdoor photophobia is reduced at least within the first 2 hours following the ocular therapy compared to outdoor photophobia without treatment; and burning sensation is reduced at least within the first 2 hours following the ocular therapy compared to burning sensation without treatment.

In certain methods, the inner portion comprises a material characterized by a first modulus; and the outer portion comprises a material characterized by a second modulus.

In certain methods, the first modulus is greater than the second modulus.

In certain methods, the first modulus is substantially the same as the second modulus.

In certain methods, the inner portion is configured to at least partially conform to the anterior surface of the cornea; the outer portion comprises at least one intermediate portion configured to at least partially conform to the anterior surface of the cornea; and the outer portion comprises at least one peripheral portion configured to engage the epithelium, the sclera, or a combination thereof, when the covering is applied to the eye.

In certain methods, a lower surface of the inner portion is characterized by at least one inner radius of curvature from 6 mm to 11 mm; a lower surface of the at least one intermediate portion is characterized by an intermediate radius of curvature from 7 mm to 9 mm; and a lower surface of the peripheral portion is characterized by a peripheral radius of curvature from 8 mm to 16 mm.

In certain methods, the lower surface of the inner portion is characterized by an inner radius of curvature corresponding to or less than a radius of curvature associated with the optical power of the cornea following the ocular therapy; and the inner portion is configured to deform during the application of the covering to the eye so that the upper surface of the inner portion optically conforms to the anterior surface of the cornea such that patient vision through the covering benefits from a post-therapy corneal profile and so as to modify tissue response of the cornea to the therapy such that, within 4 hours of the therapy, patient discomfort associated with the therapy is mitigated and/or patient vision associated with the ocular therapy is enhanced.

In certain methods, treating an eye of a patient following the ocular therapy is selected from improving post-therapy visual acuity, reducing the time to restore full visual acuity following the ocular therapy, increasing comfort, improving epithelial healing, and a combination of any of the foregoing in a population of the patients following the ocular therapy, compared to a population of patients not wearing the covering.

In certain embodiments, methods of treating an eye of a patient following cataract surgery or corneal inlay surgery, the eye comprising a cornea comprising an anterior surface, a stroma, an epithelium, and a sclera, the anterior surface of the cornea characterized by a post-surgical profile, comprise: providing a covering comprising: an inner portion comprising a first lower surface characterized by at least one inner radius of curvature, wherein the at least one inner radius of curvature comprises a radius of curvature less than a radius of curvature of the post-surgical profile of the anterior surface of the cornea, and the inner portion is characterized by a substantially uniform thickness; and an outer portion comprising a second lower surface characterized by at least one outer radius of curvature; and applying the covering, after the cataract surgery or corneal inlay surgery and prior to complete healing of the eye therefrom, wherein the covering is applied against the eye so that the covering flexes, with the inner portion at least partially disposed along and deforming so that an upper surface of the inner portion opposite the first lower surface of the inner portion optically conforms to the anterior surface of the cornea and at least a part of the outer portion engages the eye along the epithelium, the sclera, or a combination thereof.

In certain embodiments, methods of treating an eye of a patient following ocular surgery involving manipulation of the cornea comprise applying a covering to the eye of the patient following ocular surgery, wherein following treatment with the method, at least about 50% of a population of patients see 20/20 or better within about 5 minutes following application of the covering.

In certain embodiments, methods of healing a trauma wound to a cornea of an eye of a patient, the eye comprising a cornea having an anterior surface, an epithelium, and a sclera, comprise: providing a covering comprising: an inner portion characterized by an inner rigidity, and comprising an upper surface and a lower surface; and an outer portion characterized by an outer rigidity, and comprising an upper surface and a lower surface; wherein the inner rigidity is greater than the outer rigidity; applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In certain methods, the trauma is selected from physical trauma and chemical trauma.

In certain methods, the trauma is selected from blunt trauma, penetrating trauma, blast injury, burn, and psychological trauma.

In certain embodiments, methods of protecting an eye of a patient from a potential injury, the eye comprising a cornea having an anterior surface, an epithelium, and a sclera, comprise: providing a covering comprising: an inner portion characterized by an inner rigidity, and comprising an upper surface and a lower surface; and an outer portion characterized by an outer rigidity, and comprising an upper surface and a lower surface; wherein the inner rigidity is greater than the outer rigidity; applying the covering against the eye prior to the potential injury so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In certain methods, the potential injury is selected from protection from physical trauma, chemical trauma, exposure to particulates, and edema.

In certain embodiments, methods of treating an eye of a patient following cataract surgery, the eye comprising ocular tissue including a cornea having an anterior surface and an optical region used for imaging with the eye, an epithelium, and a sclera, the cataract surgery involving manipulation of the ocular tissue and associated with a lesion external to the optical region, comprise: providing a covering comprising: an inner portion characterized by an inner rigidity, and comprising an upper surface and a lower surface; and an outer portion characterized by an outer rigidity, and comprising an upper surface and a lower surface; wherein the inner rigidity is greater than the outer rigidity; following the cataract surgery, applying the covering against the eye so that the covering flexes with the lower surface of the inner portion disposed along the anterior surface of the cornea and at least partially conforming to the anterior surface; and resisting movement of the inner portion by engaging the lower surface of the outer portion with the eye along the epithelium, the sclera, or a combination thereof.

In certain methods, the inner portion optically conforms to the optical portion of the cornea.

In certain methods, the cataract surgery comprises incising the cornea and/or perforating the cornea at a site external to the optical region, and wherein the covering extends over the site.

In certain methods, the inner portion provides a smooth optical surface and the covering reduces edema.

In certain methods, the covering, when applied to the anterior surface of the cornea, seals the anterior surface of the cornea.

In certain methods, the lower surface of the inner portion is characterized by at least one radius of curvature and the at least one inner radius of curvature comprises a radius of curvature corresponding to or less than a radius of curvature associated with the optical power of the stroma following cataract surgery; and the methods further comprise deforming the inner portion during the applying of the covering so that the upper surface of the inner portion optically conforms to the anterior surface of the cornea such that patient vision through the covering benefits from the cataract surgery such that, within 4 hours of the cataract surgery, patient discomfort associated with the cataract surgery is mitigated and/or patient vision associated with the cataract surgery is enhanced.

In certain methods, the outer portion comprises a peripheral portion and one or more intermediate portions, each of the one or more intermediate portions independently characterized by an intermediate radius of curvature; and further comprising inhibiting movement of the inner portion relative to the eye by deforming at least the peripheral portion during the applying of the covering so as to promote motion-inhibiting engagement of the peripheral portion against the epithelium, the sclera, or a combination thereof, of the eye.

In certain methods, the inner portion is characterized by a substantially uniform thickness.

In certain methods, the inner rigidity is from 1.2E⁻⁶ Pa-m³ to 3.1E⁻³ Pa-m³ and the outer rigidity is from 5.4E⁻⁹ Pa-m³ to 1.5E⁻⁴ Pa-m³.

In certain methods, the inner portion is characterized by an index of refraction that corresponds substantially to the index of refraction of the cornea.

In certain methods, the inner portion and the outer portion comprise a material selected from silicone, a silicone hydrogel, or a combination thereof.

In certain methods, following treatment with the method, at least about 50% of a population of patients see 20/20 or better within about 5 minutes following the cataract surgery.

In certain methods, in a population of patients, following use of the method, treatment is characterized by at least one of: stromal edema is reduced by at least about 25 μm within about 30 minutes following the cataract surgery compared to stromal edema without treatment; overall discomfort is reduced at least within the first 2 hours following the cataract surgery compared to overall discomfort without treatment; outdoor photophobia is reduced at least within the first 2 hours following the cataract surgery compared to outdoor photophobia without treatment; and burning sensation is reduced at least within the first 2 hours following the cataract surgery compared to burning sensation without treatment.

In certain methods, the inner portion comprises a material characterized by a first modulus; the outer portion comprises a material characterized by a second modulus; and the first modulus is greater than the second modulus.

In certain methods, the inner portion is configured to at least partially conform to the anterior surface of the cornea; the outer portion comprises at least one intermediate portion configured to at least partially conform to the anterior surface of the cornea; and the outer portion comprises at least one peripheral portion configured to engage the epithelium, the sclera, or a combination thereof, when the covering is applied to the eye.

In certain methods, the lower surface of the inner portion is characterized by an inner radius of curvature corresponding to or less than a radius of curvature associated with the optical power of the cornea following the cataract surgery; and the inner portion is configured to deform during the application of the covering to the eye so that the upper surface of the inner portion optically conforms to the anterior surface of the cornea such that patient vision through the covering benefits from a post-surgical corneal profile and so as to modify tissue response of the cornea to the cataract surgery such that, within 4 hours of the cataract surgery, patient discomfort associated with the cataract surgery is mitigated and/or patient vision associated with the cataract surgery is enhanced.

In certain methods, treating an eye of a patient following the cataract surgery is selected from improving post-surgical visual acuity, reducing the time to restore full visual acuity following the cataract surgery, increasing comfort, improving epithelial healing, and a combination of any of the foregoing in a population of the patients following the cataract surgery, compared to a population of patients not wearing the covering.

In certain methods, the covering is characterized by a water content less than about 0.3%, a water permeability less than about 0.2×10⁻⁶ cm²/sec, and an oxygen permeability (Dk) from about 100 to about 500.

In certain embodiments, methods of treating an eye of a patient following cataract surgery, the eye comprising a cornea comprising an anterior surface, a stroma, an epithelium, and a sclera, the anterior surface of the cornea characterized by a post-surgical profile, comprise: providing a covering comprising: an inner portion comprising a first lower surface characterized by at least one inner radius of curvature, wherein the at least one inner radius of curvature comprises a radius of curvature less than a radius of curvature of the post-surgical profile of the anterior surface of the cornea, and the inner portion is characterized by a substantially uniform thickness; and an outer portion comprising a second lower surface characterized by at least one outer radius of curvature; and applying the covering, after the cataract surgery and prior to complete healing of the eye therefrom, wherein the covering is applied against the eye so that the covering flexes, with the inner portion at least partially disposed along and deforming so that an upper surface of the inner portion opposite the first lower surface of the inner portion optically conforms to the anterior surface of the cornea and at least a part of the outer portion engages the eye along the epithelium, the sclera, or a combination thereof.

In certain embodiments, methods of treating an eye of a patient following cataract surgery, comprise applying a covering to the eye of the patient following the cataract surgery, wherein following treatment with the method, at least about 50% of a population of patients see 20/20 or better within about 5 minutes following application of the covering.

In certain embodiments, methods of treating an eye of a patient following cataract surgery, the eye comprising ocular tissue including a cornea having an anterior surface and an optical region used for imaging with the eye, an epithelium, and a sclera, the cataract surgery involving manipulation of the ocular tissue and associated with a lesion external to the optical region, comprise: providing a covering comprising: an inner portion comprising an upper surface and a lower surface; and an outer portion comprising an upper surface and a lower surface; following the cataract surgery, applying the covering against the eye so that the lower surface of the outer portion sealingly engages the eye along the epithelium, the sclera, or a combination thereof, and so that the lower surface of the inner portion extends over the optically used portion of the cornea and along the anterior surface of the cornea.

Finally, it should be noted that there are alternative ways of implementing the embodiments disclosed herein. Accordingly, the present embodiments are to be considered as illustrative and not restrictive. Furthermore, the claims are not to be limited to the details given herein, and are entitled their full scope and equivalents thereof

What is claimed is:

1. A method of treating an eye of a patient following an ocular therapy, the eye comprising ocular tissue including a cornea having an anterior surface and an optical region used for imaging with the eye, an epithelium, and a sclera, the ocular therapy involving manipulation of the ocular tissue and associated with a lesion external to the optical region, the method comprising:
providing a covering comprising:
an inner portion characterized by an inner rigidity, and comprising an upper surface and a lower surface; and
an outer portion characterized by an outer rigidity, and comprising an upper surface and a lower surface;
wherein the inner rigidity is greater than the outer rigidity;
following the ocular therapy, applying the covering against the eye so that the covering flexes with the lower surface of the inner portion disposed along the anterior surface of the cornea and at least partially conforming to the anterior surface; and
resisting movement of the inner portion by engaging the lower surface of the outer portion with the eye along the epithelium, the sclera, or a combination thereof.

2. The method of claim 1, wherein the inner portion optically conforms to the optical portion of the cornea.

3. The method of claim 1, wherein the ocular therapy is selected from cataract surgery, corneal inlay surgery, corneal transplant surgery, and treatment of an ocular trauma wound.

4. The method of claim 1, wherein following treatment with the method, leakage from the cornea is reduced compared to leakage from the cornea without treatment.

5. The method of claim 1, wherein the inner portion provides a smooth optical surface and the covering reduces edema.

6. The method of claim 1, wherein the covering, when applied to the anterior surface of the cornea, seals the anterior surface of the cornea.

7. The method of claim 1,
wherein the outer portion comprises a peripheral portion and one or more intermediate portions, each of the one or more intermediate portions independently characterized by an intermediate radius of curvature; and
further comprising inhibiting movement of the inner portion relative to the eye by deforming at least the peripheral portion during the applying of the covering so as to promote motion-inhibiting engagement of the peripheral portion against the epithelium, the sclera, or a combination thereof, of the eye.

8. The method of claim 1, wherein the inner rigidity is from 1.2E⁻⁶ Pa-m³ to 3.1E⁻³ Pa-m³ and the outer rigidity is from 5.4E⁻⁹ Pa-m³ to 1.5E⁻⁴ Pa-m³.

9. The method of claim 1, wherein:
the inner portion comprises a material characterized by a first modulus; and
the outer portion comprises a material characterized by a second modulus.

10. The method of claim 9, wherein the first modulus is greater than the second modulus.

11. The method of claim 9, wherein the first modulus is substantially the same as the second modulus.

12. A method of healing a trauma wound to a cornea of an eye of a patient, the eye comprising a cornea having an anterior surface, an epithelium, and a sclera, the method comprising:
providing a covering comprising:
an inner portion characterized by an inner rigidity, and comprising an upper surface and a lower surface; and
an outer portion characterized by an outer rigidity, and comprising an upper surface and a lower surface;
wherein the inner rigidity is greater than the outer rigidity;
applying the covering against the eye so that the covering flexes with the lower surface of the inner portion disposed along the anterior surface of the cornea and at least partially conforming to the anterior surface; and
resisting movement of the inner portion by engaging the lower surface of the outer portion with the eye along the epithelium, the sclera, or a combination thereof.

13. A method of treating an eye of a patient following cataract surgery, the eye comprising ocular tissue including a cornea having an anterior surface and an optical region used for imaging with the eye, an epithelium, and a sclera, the cataract surgery involving manipulation of the ocular tissue and associated with a lesion external to the optical region, the method comprising:
providing a covering comprising:
an inner portion characterized by an inner rigidity, and comprising an upper surface and a lower surface; and
an outer portion characterized by an outer rigidity, and comprising an upper surface and a lower surface;
wherein the inner rigidity is greater than the outer rigidity;
following the cataract surgery, applying the covering against the eye so that the covering flexes with the lower surface of the inner portion disposed along the anterior surface of the cornea and at least partially conforming to the anterior surface; and
resisting movement of the inner portion by engaging the lower surface of the outer portion with the eye along the epithelium, the sclera, or a combination thereof.

14. The method of claim 13 wherein the inner portion optically conforms to the optical portion of the cornea.

15. The method of claim 13, wherein the inner portion provides a smooth optical surface and the covering reduces edema.

16. The method of claim 13, wherein the covering, when applied to the anterior surface of the cornea, seals the anterior surface of the cornea.

17. The method of claim 13, wherein the inner rigidity is from 1.2E⁻⁶ Pa-m³ to 3.1E⁻³ Pa-m³ and the outer rigidity is from 5.4E⁻⁹ Pa-m³ to 1.5E⁻⁴ Pa-m³.

18. The method of claim 13 wherein:
the inner portion is configured to at least partially conform to the anterior surface of the cornea;
the outer portion comprises at least one intermediate portion configured to at least partially conform to the anterior surface of the cornea; and
the outer portion comprises at least one peripheral portion configured to engage the epithelium, the sclera, or a combination thereof,
when the covering is applied to the eye.

19. The method of claim 13, wherein:
the lower surface of the inner portion is characterized by an inner radius of curvature corresponding to or less than a radius of curvature associated with the optical power of the cornea following the cataract surgery; and
the inner portion is configured to deform during the application of the covering to the eye so that the upper surface of the inner portion optically conforms to the anterior surface of the cornea such that patient vision through the covering benefits from a post-surgical corneal profile and so as to modify tissue response of the cornea to the cataract surgery such that, within 4 hours of the cataract surgery, patient discomfort associated with the cataract surgery is mitigated and/or patient vision associated with the cataract surgery is enhanced.

20. The method of claim 13, wherein the covering is characterized by a water content less than about 0.3%, a water permeability less than about 0.2×10⁻⁶ cm²/sec, and an oxygen permeability (Dk) from about 100 to about 500.
